SEQUENCE LISTING (4 sequences)

SEQ ID NO: 1 - DNA, 20 bp, Artificial Sequence
  Description of Artificial Sequence sense primer pKanEF
actcaccgaggcagttccat

SEQ ID NO: 2 - DNA, 20 bp, Artificial Sequence
  Description of Artificial Sequence antisense primer pKanER
ggtagcgttgccaatgatgt

SEQ ID NO: 3 - DNA, 20 bp, Artificial Sequence
  Description of Artificial Sequence sense primer pKanIF
atggcaagatcctggtatcg

SEQ ID NO: 4 - DNA, 20 bp, Artificial Sequence
  Description of Artificial Sequence antisense primer pKanIR
ttatgcctcttccgaccatc

(19) United States
(12) Patent Application Publication
(10) Pub. No.: US 2008/0112915 A1
(43) Pub. Date: May 15, 2008

(54) Dna Delivery with Gemini Cationic Surfactants

(76) Inventors: Marianna Foldvari, Saskatchewan (CA); Ronald E. Verrall, Saskatchewan (CA); Ildiko Badea, Saskatchewan (CA)

Correspondence Address: PERKINS COIE LLP, P.O. BOX 2168, MENLO PARK, CA, 94026, US

(21) Appl. No.: 10/577,025

(22) PCT Filed: Oct. 25, 2004
(86) PCT No.: PCT/CA04/01870

§ 371 Date: Mar. 5, 2007

Related U.S. Application Data
(60) Provisional application No. 60/514,197, filed on Oct. 24, 2003.

(51) Int. Cl. A61K 31/74; A61P 17/00

(52) U.S. Cl. 424/78.02

(57) ABSTRACT

The invention provides a delivery system for a biologically active agent comprising a gemini surfactant in admixture with the biologically active agent in a topical formulation for treatment of skin disorders or metabolic diseases. The invention also provides pharmaceutical compositions in topical formulations comprising the delivery system as set out above, in admixture with pharmaceutically acceptable carriers, diluents, excipients, or supplements suitable for application to the skin or mucosal membrane. Methods of preparation and use are also described.

### FIELD OF THE INVENTION

The present invention relates to a delivery system for a biologically active agent comprising a gemini surfactant with the biologically active agent for treatment of skin disorders and metabolic diseases.

### BACKGROUND OF THE INVENTION

As the largest and most accessible organ of the body, skin is an ideal target for gene therapy. Current delivery methods for gene therapy include biolistic or microprojectile introduction, direct injection and electroporation. Although such methods deliver genetic material directly into the skin, they are nevertheless highly invasive. Alternatively, ex vivo delivery involves removal of a skin sample from the patient; culturing of skin cells, such as epidermal keratinocytes or dermal fibroblasts; genetically engineering such cells in vitro; and returning them in the form of a skin graft back to the patient. However, this procedure may cause unacceptable scarring and trauma to the patient.

A further delivery method for gene therapy involves the use of peptide-based gemini compounds having gene transfection properties, such as those disclosed in U.S. Pat. No. 6,693,167 to Camilleri et al., issued Feb. 17, 2004; United States Patent Application Publication No. US 2003/0119188, published Jun. 26, 2003, to Camilleri et al.; United States Patent Application Publication No. US 2004/0043939, published Mar. 4, 2004, to Camilleri et al.; and United States Patent Application Publication No. US 2004/0138139, published Jul. 15, 2004, to Camilleri et al. In these references, the peptide-based gemini compounds are stated to facilitate the transfer of other compounds of interest, such as polynucleotides, into cells for drug delivery. Following synthesis, the peptide-based gemini compounds are mixed with the compound of interest and added to cells. However, such an indirect method is potentially expensive and time-consuming, as various starting materials and synthetic peptide chemistry techniques are required to prepare the peptide-based gemini compounds even before they are useful for facilitating the transfer of the desired compound. A more direct, inexpensive approach which eliminates the need for peptide-based compounds as “facilitators” of gene transfer is thus desirable. Topical delivery of genetic material itself appears promising and could provide a more continuous supply of the protein within the skin. This approach has further advantages: i) the DNA is a more stable molecule than the protein, ii) the continuous expression of protein within the skin after topical administration limits systemic exposure; iii) topical treatment could avoid aggravating any lesions by invasive procedures; and iv) topical treatment can be self-administered by the patient. However, these advantages are contingent upon successful delivery of the DNA into the skin.

To date, plasmid DNA delivery into skin has been attempted by mechanical or electrical methods. Non-invasive delivery of naked DNA has produced limited results. Gene transfer “vectors” are currently based upon viruses, for which procedures are complex, hazardous, and expensive. Further, repeated dosing is not often possible, and success cannot be guaranteed. Nonviral approaches (e.g., plasmids or oligodeoxynucleotides) are less expensive, easily manufactured, and can be readily altered to form different combinations depending upon the intended treatment (Vogel, 2000). Further, nonviral approaches permit repeated dosing over time.

Certain types of liposomes have been shown to deliver DNA into the cell and target specific tissues in vitro and in vivo (Barron et al., 1999; Xu et al., 1999; Birchall et al., 2000; Delepine et al., 2000; Babiuk et al., 2002). Liposomes lack the immunogenicity and hazards associated with viral approaches and allow introduction of larger DNA fragments into target cells. The transfection efficiency is based on physical and chemical characteristics of the building elements of the liposomes.

A gemini surfactant is a surfactant molecule which contains more than one hydrophobic tail. Each hydrophobic tail has a hydrophilic head (Menger and Keiper, 2000; Kirby et al., 2003). The hydrophobic tails or hydrophilic heads are linked together by a spacer. The hydrophobic tails can be identical or differ. Likewise, the hydrophilic heads can be identical or differ. Further, the hydrophilic heads may be anionic (e.g. of a phosphate, sulphate or carboxylate type), cationic (e.g. of a quaternary ammonium type), nonionic or neutral (e.g. of a polyether, peptide or sugar type), or amphoteric (Menger and Keiper, 2000). In aqueous solutions, gemini surfactants spontaneously aggregate into micelles whose shape and size are particularly sensitive to the length and hydrophobic or hydrophilic nature of the spacer. The spacer can be variable, namely short (e.g., 2 methylene groups) or long (e.g., more than 12 methylene groups); rigid (e.g., stilbene) or flexible (e.g., methylene chain); and polar (e.g., polyether, ethoxyl, polyethoxyl) or nonpolar (e.g., aliphatic, aromatic) (Menger and Keiper, 2000). As the hydrophobic tails, hydrophilic heads and spacer can vary with regard to the above aspects, innumerable different molecules can be designed. Due to the unique physical properties arising from their structure, gemini surfactants display promise as nonviral delivery systems for biologically active agents. Significantly, topical application of gemini surfactants is most desirable for the advantages previously described. Such a system further provides ease of administration and comfort to the patient, features which are most desirable in gene therapy.

### SUMMARY OF THE INVENTION

The present invention broadly relates to a delivery system for a biologically active agent comprising:

a gemini surfactant in admixture with a biologically active agent in a topical formulation, wherein the delivery system, when in contact with the skin or mucosal membrane, releases the biologically active agent in a therapeutically-effective amount to provide a localized or systemic effect for treatment of a skin disorder or a metabolic disease.

In another broad aspect, the invention also provides a pharmaceutical composition in a topical formulation comprising:

the delivery system according to the above, in admixture with one or more pharmaceutically acceptable carriers, diluents, excipients, or supplements suitable for application to the skin or mucosal membrane.

In another aspect, the invention provides a method of treating skin disorders and metabolic diseases comprising:

contacting the skin or mucosal membrane of a subject with a delivery system comprising a gemini surfactant in admixture with a biologically active agent in a topical formulation, wherein the delivery system, when in contact with the skin or mucosal membrane, releases the biologically active agent in a therapeutically-effective amount to provide a localized or systemic effect for treatment of a skin disorder or a metabolic disease.

In another aspect, the invention provides use of a delivery system in the treatment of a skin disorder or metabolic disease, wherein the delivery system comprises a gemini surfactant in admixture with a biologically active agent in a topical formulation, and the delivery system, when in contact with the skin or mucosal membrane, releases the biologically active agent in a therapeutically effective amount to provide a localized or systemic effect.

In yet a further aspect, the invention provides use of a gemini surfactant in the manufacture of a delivery system with a biologically active agent in a topical formulation for treatment of a skin disorder or metabolic disease.

As used herein and in the claims, the terms and phrases set out below have the meanings which follow:

“Biocompatible” means generating no significant undesirable host response for the intended utility. Most preferably, biocompatible materials are non-toxic for the intended utility. Thus, for human utility, biocompatible is most preferably non-toxic to humans or human tissues.

“Carriers, diluents, excipients or supplements” as used in the pharmaceutical compositions of the present invention are meant to refer to vehicles which are biocompatible, pharmaceutically acceptable, and suitable for administration to the skin or mucosal membrane.

“Expression” means the transcription of a gene into structural RNA (rRNA, tRNA) or messenger RNA (mRNA) with subsequent translation into a protein.

“Gemini surfactant” means a surfactant molecule which contains more than one hydrophobic tail. Each hydrophobic tail has a hydrophilic head. The hydrophobic tails or hydrophilic heads are linked together by a spacer. The hydrophobic tails can be identical or differ. Likewise, the hydrophilic heads can be identical or differ. Further, the hydrophilic heads may be anionic (e.g. of a phosphate, sulphate or carboxylate type), cationic (e.g. of a quaternary ammonium type), nonionic or neutral (e.g. of a polyether, peptide or sugar type), amphoteric, or mixtures thereof, but are preferably cationic, and most preferably of a quaternary ammonium type.

“Host” or “host animal” or “subject” means humans or other vertebrates.

“Mucosal membrane” means the epithelial membranes which line the oral cavity, the nasal, bronchial, pulmonary, trachea and pharynx airways, the otic and ophthalmic surfaces, the urogenital system, including the prostate, the reproductive system and the gastrointestinal tract including the colon and rectal surfaces. The term is meant to include the surface membranes or cell structures of the mucosal membrane at a subject's targeted site.

“Pharmaceutically- or therapeutically-effective” means any amount of the delivery system or macromolecule which will exhibit the desired effect upon administration. The amount of the delivery system administered will vary with the condition being treated, the stage of advancement of the condition, the age and type of host, and the type and concentration of the formulation being applied. Appropriate amounts in any given instance will be readily apparent to those skilled in the art or capable of determination by routine experimentation.

“Pharmaceutically- or therapeutically-acceptable” means a substance which does not significantly interfere with the effectiveness or the biological activity of the active agents and which has an acceptable toxic profile for the host to which it is administered.

“Plasmid” means an extrachromosomal hereditary determinant, or a self-replicating circular molecule of DNA which is found in a variety of bacterial, archaeal, fungal, algal, and plant species.

A “polynucleotide” or “nucleic acid” means a linear sequence of deoxyribonucleotides (in DNA) or ribonucleotides (in RNA) in which the 3′ carbon of the pentose sugar of one nucleotide is linked to the 5′ carbon of the pentose sugar of the adjacent nucleotide via a phosphate group. The “polypeptide” or “nucleic acid” may comprise DNA, including cDNA, genomic DNA, and synthetic DNA, or RNA, which may be double-stranded or single-stranded, and if single-stranded, may be the coding strand or non-coding (anti-sense) strand.

A “protein” or “polypeptide” means a linear polymer of amino acids that are linked by peptide bonds.

“Topical,” as in a topical formulation, is meant to refer to formulations for any area of the skin or mucosal membrane.

“Transfection” means the integration of foreign DNA into the genome of a host cell via direct gene transfer.

A “vector” means a nucleic acid molecule that is able to replicate autonomously in a host cell and can accept foreign DNA.

### BRIEF DESCRIPTION OF THE DRAWINGS

FIG. 1 shows the general structure of gemini surfactants.

FIG. 2A is a schematic illustration of the structure of DNA-gemini surfactant-lipid complexes used for transfection.

FIG. 2B is a schematic illustration of the structure of cationic gemini liposomal formulations.

FIG. 2C is a schematic illustration of the structure of gemini nanoemulsion formulations.

FIG. 3A shows size measurements for the transfection mixture prepared with the 16-3-16 gemini surfactant.

FIG. 3B shows size measurements for the transfection mixture prepared with the 16-3-16 gemini surfactant.

FIG. 3C shows size measurements for the topical nanoemulsion formulation prepared with the 16-3-16 gemini surfactant.

FIG. 4 is a graph showing IFNγ expression in PAM212 cells transfected with 0.2 μg pIRES.GFP, pIRES.IFN-GFP, pGTmCMV and pGTmCMV.IFN-GFP, using Lipofectamine Plus™ Reagent.

FIG. 5A is a graph showing INFγ expression in PAM 212 keratinocytes assessed by GFP expression.

FIG. 5B is a graph showing INFγ expression in PAM 212 keratinocytes assessed by cell viability by FACS.

FIG. 6 is a graph showing the influence of transfection duration on the efficiency of the transfection.

FIG. 7A illustrates the circular dichroism spectra of plasmid-gemini complexes.

FIG. 7B illustrates the circular dichroism spectra of plasmid-gemini-DOPE liposomal complexes.

FIG. 8A is a graph showing IFNγ expression in skin in mice treated with the pGTmCMV.IFN-GFP plasmid and gemini lipid 16-3-16 in various formulations.

FIG. 8B is a graph showing IFNγ expression in lymph nodes in mice treated with the pGTmCMV.IFN-GFP plasmid and gemini lipid 16-3-16 in various formulations.

### DETAILED DESCRIPTION OF THE INVENTION

### A. Preparation

Preparation of the delivery system can involve initial preparation of the gemini surfactants with the biologically active agent of interest to form the gemini surfactant-biologically active agent complex, and using the gemini surfactant-biologically active agent complex alone or in combination with suitable supplements to provide formulations for administration to a subject for use in treatment of skin disorders and metabolic diseases. Alternatively, the gemini surfactant does not have to be combined with the biologically active agent first, but can be combined with suitable supplements prior to preparation of the gemini surfactant-biologically active agent complex. The gemini surfactant can thus be combined in any order with the biologically active agent.

i. Gemini Surfactants

A gemini surfactant is a surfactant molecule which contains more than one hydrophobic tail. Each hydrophobic tail has a hydrophilic head (Menger and Keiper, 2000; Kirby et al., 2003). The hydrophobic tails or hydrophilic heads are linked together by a spacer. The hydrophobic tails can be identical or differ. Likewise, the hydrophilic heads can be identical or differ. Further, the hydrophilic heads may be anionic (e.g. of a phosphate, sulphate or carboxylate type), cationic (e.g. of a quaternary ammonium type), nonionic or neutral (e.g. of a polyether, peptide or sugar type) or amphoteric (Menger and Keiper, 2000). In aqueous solutions, gemini surfactants spontaneously aggregate into micelles whose shape and size are particularly sensitive to the length and hydrophobic or hydrophilic nature of the spacer. The spacer can be variable, namely short (e.g., 2 methylene groups) or long (e.g., more than 12 methylene groups); rigid (e.g., stilbene) or flexible (e.g., methylene chain); and polar (e.g., polyether, ethoxyl or polyethoxyl) or nonpolar (e.g., aliphatic, aromatic) (Menger and Keiper, 2000). As the hydrophobic tails, hydrophilic heads and spacer can vary with regard to the above aspects, innumerable different molecules can be designed

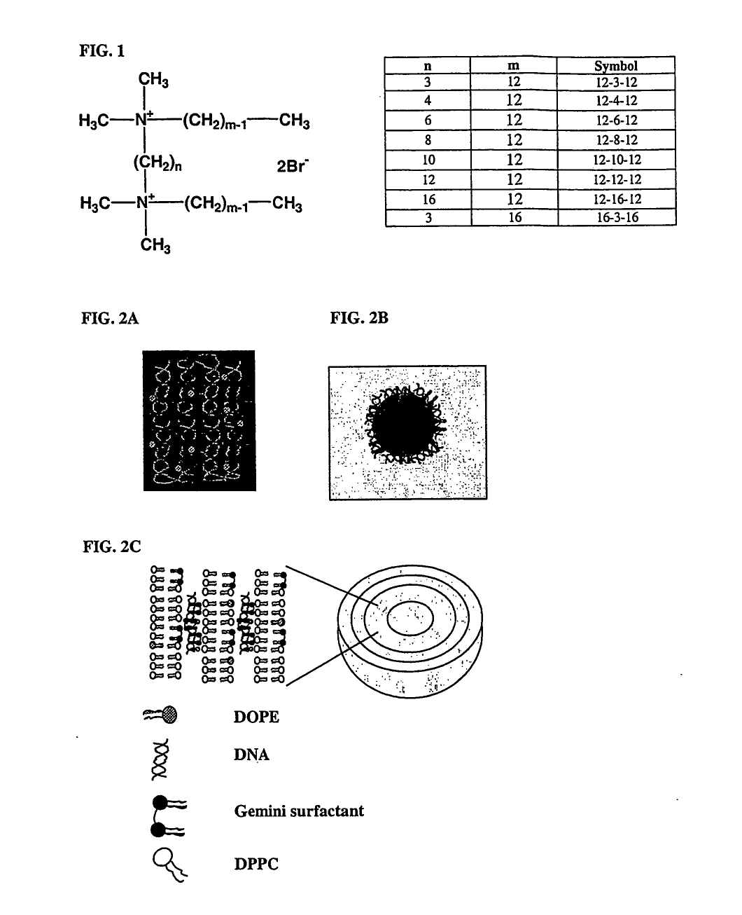

The general structure of a gemini surfactant is shown in FIG. 1 to include a head group composed of two positively charged nitrogen atoms, separated by a spacer (n) of 3, 4, 6, 8, 10, 12, or 16 carbon atoms and each containing two methyl groups, and the tails consist of two saturated 12 or 16 carbon atom chains (m=12 or 16), respectively.

For the present invention, the exemplary type of hydrophobic tail is a C₃-C₃₀ alkyl group, linear or branched, saturated or unsaturated. Although the hydrophilic heads may be anionic, cationic, neutral or amphoteric, the hydrophilic heads are preferably cationic in the present invention. Gemini cationic surfactants are capable of compacting DNA and have several advantages compared to the classic monovalent surfactants, for example, lower cellular toxicity, low critical micellar concentration and greater tendency to self-assemble, and greater variety due to the various spacers separating the two quaternary nitrogen atoms. This variety contributes to the flexibility in designing suitable delivery systems for different target cells.

Among the vast classes of gemini surfactants, the types preferable for use in the invention are those with linear hydrocarbon tailgroups and quaternary ammonium headgroups.

Selection of the particular gemini surfactant is significant, in that the magnitude of transfection is significantly dependent on the spacer length of the selected gemini cationic surfactants. A larger Gibbs area (a) per molecule (lower surface excess concentration, Γ, at the air/water interface) (Wettig et al., 2001) correlates with a lower transfection efficiency. These parameters reflect the importance of the spacer length in binding of the gemini cationic surfactant to DNA. The distance between two phosphate groups in a DNA molecule is 0.34 nm, whereas the distance between the cationic head groups in gemini cationic surfactants 12-3-12 and 12-6-12 are estimated to be 0.49 and 0.91 nm, respectively (Chen et al., 2002). Thus, when the distance between head groups in gemini cationic surfactants approaches that between phosphate groups in DNA, stronger complexation may occur.

There is also evidence that longer spacers will bend into a U shape and preferentially locate in a more hydrophobic environment. This U shape formation and the resulting decrease in distance between the two cationic head groups are apparent at spacer lengths greater than twelve carbon atoms (Alami et al., 1993; Chen et al., 2002).

Gemini surfactants can be prepared from readily available starting materials using synthetic chemistry known to those skilled in the art, as reviewed by Menger and Keiper (2000).

ii. Biologically Active Agents

Biologically active agents which can be used with the present invention include, but are not limited to, nucleic acids, plasmid DNA, DNA vaccines, proteins, vaccines, immunoglobulins, immunomodulators, oligonucleotides, peptides, hormones, toxins, and enzymes. Most preferably, the biologically active agents for use with the present invention are nucleic acids, plasmid DNA, DNA vaccines, and oligonucleotides.

iii. Supplements

Various supplements can be used to enhance the transfection efficiency. Such supplements generally promote the formation of liposomes around the gemini surfactant-biologically active agent complex. Liposomes are microscopic vesicles containing phospholipid bilayers which enclose aqueous spaces. In a formulation, liposomes carry both water and oil soluble payloads, can solubilize recalcitrant compounds, prevent oxidation, stabilize proteins, and control hydration. Liposomes hold normally immiscible materials together in a microsphere with controllable release of the encapsulated ingredients. For formulations containing the gemini surfactant-biologically active agent complex of the invention, suitable supplements include, but are not limited to:
a) a neutral carrier, such as dioleyl phosphatidylethanolamine (DOPE) which is a nonionic “helper lipid,” or cholesterol; and
b) permeation enhancers, for example, Transcutol™ (diethylene glycol monoethyl ether), propylene glycol, oleic acid, and terpenes. There are more than 300 known permeation enhancers, which belong to one of three essential groups based upon their mechanism of permeation enhancement (Williams and Barry, 1992; Chattaraj and Walker, 1995). Group 1 permeation enhancers are capable of extracting skin lipids or damaging the stratum corneum, hence weakening barriers to permeation (e.g., solvents such as ethanol and organic acids such as salicylic acid). Further, Group 2 permeation enhancers increase the solubility of the biologically active agent within the skin (e.g. propylene glycol). Lastly, Group 3 permeation enhancers perturb intercellular lipids (e.g., terpenes, surfactants, fatty acids, fatty acid esters, Azone and derivatives, amides such as dimethylformamide, and sulfoxides such as DMSO).

### B. Formation of the Gemini Surfactant-Biologically Active Agent Complex

Gemini surfactants can be prepared from readily available starting materials using synthetic chemistry known to those skilled in the art (Menger and Keiper, 2000). Biologically active agents (i.e., nucleic acids, plasmid DNA, DNA vaccines, proteins, vaccines, immunoglobulins, immunomodulators, oligonucleotides, peptides, hormones, toxins, and enzymes) can be prepared using techniques known to those skilled in the art (see, for example, Ausubel et al., 2000; Sambrook et al., 1989) before combining with the gemini surfactant to form the gemini surfactant-biologically active agent complex.

In the Examples, the invention is demonstrated using a gene as the biologically active agent. The gene encoding for murine INFγ is inserted as part of the plasmid. Briefly, a suitable plasmid is constructed to include the gene encoding the protein of interest, and control sequences such as promoters, enhancers, and terminators, with signal sequences and selectable markers included if desired; for instance, in the Examples, a murine CMV promoter and a GFP gene were included for easy qualitative evaluation of protein expression. The IFNγ gene was inserted into the multiple cloning site with the GFP in a bicistronic format. Such considerations are important, since the level of IFNγ expression was found to be 20 times higher when using the pGTmCMV.IFN-GFP than with the pIRES.IFN-GFP plasmid.

The vector is preferably one which is specifically designed for gene therapy, and is incapable of inducing an immune response; for instance, in the Examples, the vector lacks CpG motifs. The vector should be able to replicate autonomously in a host cell and accept foreign DNA. A vector carries its own origin of replication, one or more unique recognition sites for restriction endonucleases which can be used for the insertion of foreign DNA, and often recognition sequences (e.g. promoter) for the expression of the inserted DNA. Any vector may be used as long as it is replicable and viable in the host.

The gemini surfactant and biologically active agent are combined to form the gemini surfactant-biologically active agent complex using techniques known in the art (see, for example, Ausubel et al., 2000; Sambrook et al., 1989). In the Examples, the constructed plasmid is simply mixed with aqueous gemini cationic surfactant to obtain the gemini cationic surfactant-DNA complex.

Preparation of the delivery system can involve initial preparation of the gemini surfactants with the biologically active agent of interest to form the gemini surfactant-biologically active agent complex, and using the gemini surfactant-biologically active agent complex alone or in combination with suitable supplements to provide formulations for administration to a subject for use in treatment of skin disorders and metabolic diseases. Alternatively, the gemini surfactant does not have to be combined with the biologically active agent first, but can be combined with suitable supplements prior to preparation of the gemini surfactant-biologically active agent complex. The gemini surfactant can thus be combined in any order with the biologically active agent.

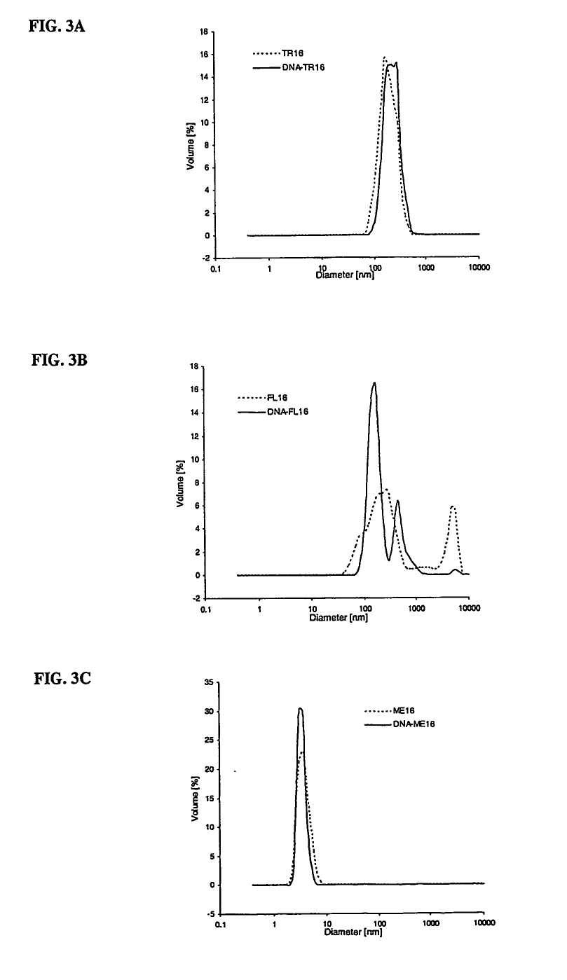

The gemini cationic surfactant-DNA complex can be used alone or incorporated into topical formulations. FIGS. 2A, 2B and 2C are schematic illustrations of the structure of DNA-gemini surfactant-lipid complexes used for transfection, cationic gemini liposomal formulations, and gemini nanoemulsion formulations, respectively. The structure and size measurements (FIGS. 3A, 3B and 3C) of several formulations were determined. Two formulations are presented in Example 4. For one formulation (the cationic liposomal formulation), DOPE, DPPC, Transcutol™ and the gemini surfactant, containing a desired concentration of the biologically active agent (e.g., plasmid) is prepared. The supplements assist in formation of liposomes. Formation of the liposome assists in compacting the highly negatively charged DNA into a dense, positively charged or neutral particle small enough to be taken up by the cells. This generally is achieved by use of a highly positively charged compound to neutralize the negative charges of the DNA.

For a second formulation (the cationic nanoemulsion), the nanoemulsion is prepared by combining a surfactant, co-surfactant, oily phase component, and the gemini surfactant, containing the biologically active agent (e.g., plasmid) at a desired concentration. PEG-8 caprylic/capric glycerides or other suitable surfactants known in the art can be used. Suitable co-surfactants can include, but are not limited to, polyglyceryl 3-diisostearate, polyglyceryl-6 isostearate, and polyglyceryl-6 dioleate. Suitable oily phase components can be selected from propylene glycol monocaprylate, oleoyl macrogol-6 glycerides, PEG-8 glyceryl linoleate, propylene glycol laurate, and propylene glycol monolaurate. As an example of a cationic nanoemulsion, the gemini cationic surfactant-DNA complex is combined with PEG-8 caprylic/capric glycerids as the surfactant; polyglycerol-3-isosterate as the co-surfactant; and octyldodecyl myristate as the oily phase as described in Example 4.

The lipid formulations can be optimized for plasmid DNA:gemini cationic surfactant charge ratio as well known by those skilled in the art. As previously discussed, the magnitude of transfection is significantly dependent on the spacer length of the selected gemini surfactants. A larger Gibbs area (a) per molecule (lower surface excess concentration, Γ, at the air/water interface) (Wettig et al., 2001) correlates with a lower transfection efficiency. These parameters reflect the importance of the spacer length in binding of the gemini cationic surfactant to DNA. The distance between two phosphate groups in a DNA molecule is 0.34 nm, whereas the distance between the cationic head groups in gemini cationic surfactants 12-3-12 and 12-6-12 are estimated to be 0.49 and 0.91 nm, respectively (Chen et al., 2002). Thus, when the distance between head groups in gemini cationic surfactants approaches that between phosphate groups in DNA, stronger complexation may occur.

There is also evidence that longer spacers will bend into a U shape and preferentially locate in a more hydrophobic environment. This U shape formation and the resulting decrease in distance between the two cationic head groups are apparent at spacer lengths greater than twelve carbon atoms (Alami et al., 1993; Chen et al., 2002).

As shown in Example 3, four different plasmid DNA:gemini cationic surfactant charge ratios were tested, and the optimal plasmid DNA:gemini cationic surfactant charge ratio was determined by comparing the quantity of expressed IFNγ with the number of fluorescent cells and determining cell viability. In general, a greater than optimal plasmid DNA:gemini cationic surfactant charge ratio results in lower cell viability and lower expression of the protein.

Further, the transfection efficiency of the gemini cationic surfactants can be determined by correlating the physico-chemical characteristics of the gemini cationic surfactants with the expression of the gene of interest. In Example 3, eight gemini cationic surfactants were tested to determine the effect of head group spacer length and alkyl chain length on their transfection efficiency. In this Example, the transfection efficiency was found to be dependent on the length of the spacer between the two positively charged head groups, with the C3 spacer showing the highest activity.

### C. Formulations, Dosages, and Treatment

The invention provides a method of delivering biologically active agents by preparing the delivery system (the gemini surfactant-biologically active agent complex as described above) and administering the delivery system topically to the skin or mucosal membrane. Most preferably, the biologically active agents for use with the present invention are nucleic acids, plasmid DNA, DNA vaccines, and oligonucleotides. Further, the delivery system can be used for localized (intradermal and intramucosal), or systemic (transdermal or transmucosal) delivery, as well as for sustained release in or beneath the skin or mucosal membrane, namely, the epithelial membranes which line the oral cavity, the nasal, bronchial, pulmonary, trachea and pharynx airways; the otic and ophthalmic surfaces; the urogenital system, including the prostate, the reproductive system; the gastrointestinal tract including the colon and rectal surfaces; and the surface membranes or cell structures of the mucosal membrane at a subject's targeted site.

For this purpose, various formulations can be used for administration of the delivery system to the skin or mucosal membrane. Such formulations, whether pharmaceutically acceptable preparations or devices, preferably maintain contact with the skin or mucosal membrane. As formulations of the delivery system may lose some activity with aging, they can be either stabilized or generated fresh for administration.
Creams, Lotions, Pastes, Ointments, Foams—The delivery system may be incorporated into lipid formulations, emulsions, suspensions, creams, lotions, pastes, ointments or foams. Ointments or creams can be formulated with an aqueous or oily base with the addition of suitable thickening and/or gelling agents. Such bases may include water and/or an oil such as liquid paraffin or a vegetable oil such as peanut oil or castor oil. An exemplary base is water. Thickening agents which can be used according to the nature of the base include aluminum stearate, hydrogenated lanolin, and the like. Further, lotions can be formulated with an aqueous base and will, in general, include one or more of the following: stabilizing agents, emulsifying agents, dispersing agents, suspending agents, thickening agents, coloring agents, perfumes, and the like. Ointments and creams can also contain excipients, such as starch, tragacanth, cellulose derivative, polyethylene glycols, silicones, bentonites, silicic acid, and talc, or mixtures thereof. Lotions may be formulated with an aqueous or oily base and will, in general, also include one or more of the following: stabilizing agents, emulsifying agents, dispersing agents, suspending agents, thickening agents, coloring agents, perfumes, and the like. Foams may be formed with known foaming or surface active agents.
Gels and Liquids—The delivery system may be incorporated into gels, aqueous or non-aqueous solutions, sprays, mists or aerosols. Gels may be formed by mixing the delivery system with gelling agents such as collagen, pectin, gelatin, agarose, chitin, chito san and alginate. The delivery system may be incorporated into liquids, formulated as topical solutions, aerosols, mists, sprays, drops and instillation solutions for body cavities. Administration of the delivery system to the mucosal membrane may be performed by aerosol, which can be generated by a nebulizer, or by instillation.
Coated Substrates—Substrates such as dressings, packings, films or meshes can be coated with the delivery system and used directly on the skin or mucosal membrane.
Transdermal Patch—Transdermal patches incorporating the delivery system can be attached to the skin or mucosal membrane to provide controlled, sustained release of the biologically active agent in or within the skin or mucosal membrane.

The delivery system may be administered alone, or with suitable non-toxic, pharmaceutically acceptable carriers, diluents and excipients suitable for topical application, as are well known in the art, see for example, Merck Index, Merck & Co., Rahway, N.J.; and Gilman et al., (eds) (1996) Goodman and Gilman's: The Pharmacological Bases of Therapeutics, 10^{th} Ed., McGraw-Hill. Carriers, diluents, excipients or supplements as used in the pharmaceutical compositions of the present invention are meant to refer to vehicles which are biocompatible, pharmaceutically acceptable, and suitable for administration to the skin or mucosal membrane. For standard dosages of conventional pharmacological agents, see for example, the U.S. Pharmacopeia National Formulary (2003), U.S. Pharmacopeial Convention, Inc., Rockville, Md. All agents must be non-toxic and physiologically acceptable for the intended purpose, and must not substantially interfere with the activity of the biologically active agent.

The dosage of the delivery system depends upon many factors that are well known to those skilled in the art, for example, the particular form of the biologically active agent within the delivery system, the condition being treated, the age, weight, and clinical condition of the recipient patient, and the experience and judgement of the clinician or practitioner administering the therapy. A therapeutically effective amount provides either subjective relief of symptoms or an objectively identifiable improvement as noted by the clinician or other qualified observer. The dosing range varies with the biologically active agent within the delivery system used, its form, and the potency of the particular agent.

To demonstrate the preparation and method of use of the delivery system of the present invention, the inventors evaluated the effectiveness of the delivery system in topical delivery of the gene coding for IFNγ as potential therapy for treatment of scleroderma. Scleroderma is a complex disease that is classified into two major groups, namely the types that affect the skin only (localized scleroderma: morphoea and linear scleroderma) and types where in addition to skin, internal organs (esophagus, gastrointestinal tract, lungs, kidneys, heart and muscles) are involved (systemic sclerosis: diffuse, limited and other) (Moschella and Hurley, 1992). Scleroderma is an autoimmune connective tissue disease in which, for unknown reasons, the skin becomes thick and hard due to the excessive production and deposition of collagen. Similar changes can occur in the internal organs as well. Other clinical manifestations include pain and stiffness of joints, abnormal sensitivity to cold in the extremities (Raynaud's syndrome), swelling of hands and feet, oral, facial and dental problems, among others. Therapeutic options for this disease are limited and the clinical progress of the disease is largely uncontrollable. The excessive proliferation and deposition of collagen within the skin is a major pathological hallmark of the disease.

T-cell derived IFNγ is one of the most potent inhibitors of collagen gene transcription in fibroblasts. In vitro studies clearly showed that IFNγ reduces fibroblast collagen synthesis, induces the repression of fibroblast growth and modulates the interactions between cells and intercellular matrix that can lead to a more optimized collagen network (Harrop et al., 1995; Widom, 2000). A model of the molecular pathology of scleroderma shows that IFNγ gene therapy may have an effect on three groups of pathophysiological markers of scleroderma, namely cytokines (1); collagen and extracellular matrix components (2), and cell adhesion molecules (3) (compiled from Bos et al., 1997; Luger et al., 1997; Galperin and Gershwin, 1998; Arnett, 2002). Based on this pathogenesis model, it can be speculated that the administration of IFNγ could also have an indirect inhibitory effect on TGF-β and an immunomodulatory effect on T cells to switch the Th1/Th2 balance toward Th1. The limitation of the treatment by IFNγ is related to the non-targeted administration method. Subcutaneous or intramuscular injection of IFNγ does not provide sufficient levels of this cytokine within the specific target areas of the skin; therefore, the main challenge is the delivery and targeting of IFNγ to the epidermal and dermal layers of the skin.

The inventors addressed this problem using the delivery system of the present invention. Briefly, a suitable plasmid was constructed. Transfection and cellular expression of IFNγ from pGTmCMV.IFN-GFP plasmid were evaluated in PAM212 keratinocyte culture. The plasmid/gemini cationic surfactant (varying spacer and chain lengths) complexes were characterized by circular dichroism and microscopy. The in vitro transfection efficiency was found to be dependent on the spacer length of the gemini surfactant. For topical formulations, the inventors prepared two delivery systems, namely a cationic liposomal formulation and a cationic nanoemulsion, both of which incorporate a cationic gemini surfactant. Mice were treated topically with such formulations, and the INFγ expression was evaluated, showing high levels of IFNγ expression in the skin and lymph nodes. The inventors thus found that the delivery system of the present invention demonstrates effectiveness as a topical form of gene therapy.

Although the invention is described herein for treatment of scleroderma, it will be appreciated that the invention is equally useful for the therapeutic treatment of other conditions characterized by any cytokine deficiency or IFNγ deficiency. Atopic dermatitis, for example, is a chronic pruritic eruption occurring in adolescents and adults, of unknown etiology although allergic, hereditary, and psychogenic factors appear to be involved (Friel, 1974). Staphylococcus aureus is believed to contribute to exacerbations of the disease. The lesions occur chiefly on the knees and elbows, but may involve other areas such as the face and arms, and are marked by lichenification, excoriations, and crusting. The disease is associated with diminished IFNγ synthesis (Katsunuma et al., 2004). As the invention has been demonstrated in the Examples using the gene coding for IFNγ for treatment of scleroderma, it will be appreciated that the invention can be extended to treatment of atopic dermatitis or any other condition characterized by IFNγ deficiency, upon which the delivery of the gene coding for IFNγ may have ameliorative effects.

The delivery system can thus be used for localized delivery to treat skin disorders, particularly atopic dermatitis, psoriasis, conditions characterized by any cytokine deficiency, conditions characterized by IFNγ deficiency, genodermatoses (skin diseases of genetic origin) including epidermal fragility disorders, keratinization disorders, hair disorders, pigmentation disorders, porphyrias, multisystem disorders and cancer disorders, as reviewed by Uitto and Pulkkinen (2000). For example, candidate diseases for treatment with the present invention include, but are not limited to, forms of inherited epidermolysis bullosa (such as junctional EB and dystrophic EB which are characterized by extreme fragility of the skin and mucosal membrane); lamellar ichthyosis and X-linked ichthyosis (characterized by epithelial cornification and defective skin barrier function); and xeroderma pigmentosum (characterized by defective DNA repair functions leading to severe blistering upon exposure to sunlight and development of multiple skin tumours).

Further, the delivery system can be used for systemic delivery to treat metabolic diseases through modification of epidermal keratinocytes within the skin. Epidermal keratinocytes normally secrete factors into the extracellular matrix which then reach the bloodstream (Spirito et al., 2000). Such metabolic diseases which could be amenable to treatment using the delivery system of the present invention include, but are not limited to, conditions characterized by any cytokine deficiency, conditions characterized by IFNγ deficiency, gyrate atrophy, maternal hyperphenylalaninemia, familial hypercholesterolemia, and phenylketonuria.

It will be further appreciated that the delivery system can be used with any gene having therapeutic effects for the above skin disorders and metabolic diseases.

Abbreviations and nomenclature employed herein are standard in the art and are commonly used in scientific publications such as those cited herein. The invention is further illustrated by the following non-limiting examples.

### D. EXAMPLES

### Example 1

### Preparation of Plasmids

The pGT is a vector designed for gene therapy. It contains the human cytomegalovirus (CMV) promoter, having the CpG motifs removed, where the human CMV was replaced with the murine CMV (Dorsch-Häsler et al, 1985) to give the pGTmCMV backbone. The IFNγ gene (Gray et al., 1983) was obtained from the pSLRSV.IFN plasmid (Lewis et al., 1997) that contained the sequence coding for 155 amino acids of the murine IFNγ. The plasmid pIRES2-EGFP (Clontech, Palo Alto, Calif.) contains the gene encoding for the enhanced green fluorescent protein fused with IRES sequence, preceded by a multiple cloning site for gene insertion. The gene encoding for murine IFNγ was inserted at the Bgl II site, creating the plasmid pIRES.IFN-GFP. The pGTmCMV.IFN-GFP was constructed by inserting the IFNγ-IRES-GFP fragment into Bgl II and Xba I sites of the pGTmCMV vector. The plasmids were purified using QIAGEN Plasmid Purification Kit (Qiagen, Mississauga, ON). The gene of IFNγ and IFNγ-IRES-GFP fragment were sequenced (PBI/NRC, Saskatoon, SK) and compared with sequences retrieved from GenBank (NCBI databases). Reagents were purchased from Invitrogen Life Technologies (Carlsbad, Calif.) and restriction enzymes from Amersham Pharmacia Biotech (Baie d'Urfe, QB).

### Example 2

### Transfection of PAM212 Cells with Plasmid Constructs, Using Lipofectamine Plus™ Reagent

PAM 212 murine keratinocyte cells (provided by Dr. S. Yuspa, NCL Bethesda, Mass.) were grown to 90% confluency in 75-cm² tissue culture flasks in supplemented MEM, prepared from minimal essential media (MEM) (GIBCO BRL, Burlington ON) with Antibiotic Antimycotic Solution (Sigma, Burlington, ON) 1:100 dilution, and 10% v/v fetal bovine serum (FBS) (Cansera, Etobicoke ON). The day before transfection 5×10⁴ cells/well were seeded on 24-well plates (Greiner Labortechnik GmbH, Germany), on 13-cm diameter cover slips (CANEMCO, St. Laurent, QB). The plates were incubated overnight at 37° C. in a CO₂ incubator to 70-80% confluency. The supplemented MEM was changed to MEM one hour prior to transfection. The cells were transfected with the following plasmids: pIRES, pIRES.IFN-GFP, pGTmCMV, and pGTmCMV.IFN-GFP, using Lipofectamine Plus™ Reagent (Invitrogen Life Technologies, Carlsbad, Calif.). For each well 0.2 μg of plasmid was used. The transfection method followed the manufacturer's protocol and was optimized for the PAM 212 cells. Briefly, 0.2 μg of plasmid was mixed with 10 μL of PLUS reagent in 25 μL MEM and incubated at room temperature for 15 minutes. Four μL Lipofectamine, mixed with 25 μL MEM, was added to the plasmid. After incubating the mixture for 15 minutes at room temperature, it was added dropwise to cells that were covered with 200 μL of fresh MEM. The plates were incubated for 5 hours at 37° C. in a CO₂ incubator (Sanyo Electric Co. Ltd., Japan), then the transfection mix was replaced with supplemented MEM, and the incubation was continued for 24 h, after which the supernatants were collected. Media on the cells was replaced with fresh media, and after another 24-hour incubation period, the second supernatants were collected and stored at −20° C.

### Example 3

### Transfection of PAM212 Cells with pGTmCMV.IFN-GFP Plasmid Using Gemini Cationic Surfactants and DOPE

The pGTmCMV.IFN-GFP plasmid was used at a concentration of 0.2 μg/well for transfection. Eight gemini surfactants were tested in this study (FIG. 1) (Wettig et al., 2001). Aqueous solutions (1.5 mM) of the gemini cationic surfactants were prepared and filtered through 0.2 μm Acrodisc® filters (Pall Gelman, Ann Arbor, Mich.). Lipid vesicles were prepared by using sonication techniques. 1,2 dioleyl-sn-glycero-phosphatidylethanolamine (DOPE) (Avanti Polar Lipids, Alabaster, Ala.) and α-tocopherol (Spectrum, Gardena Calif.) in 1:0.2 weight ratios were dissolved in 100% ethanol (Commercial Alcohols Inc., Brampton, ON) at a concentration of 10 mg lipid/mL in a round bottom flask. The solvent was evaporated in a rotary evaporator (Rotavapor RE111 BÜCHI Laboratoriums-Technik AG, Switzerland) at 100 rpm and 55° C. to deposit a thin film on the walls of the vessel. To remove traces of organic solvent, the lipid was lyophilized overnight. Glass beads were added to the flask and the lipid was resuspended at 1 μmol/mL concentration in 9.25% w/v isotonic sucrose (Spectrum, Gardena, Calif.) solution (pH 9). The suspensions were bath-sonicated (Branson 2200, Cleansonic Orange, Va.) for 3 hours at 55° C., followed by filtration through 0.45 μm Acrodisc® filters. PAM 212 murine keratinocyte cells were prepared for transfection as described earlier.

The transfection mixtures were prepared as follows: 0.2 μg of plasmid was mixed with aliquots of gemini surfactant solution to obtain plasmid DNA:gemini surfactant charge ratios of 1:5, 1:10, 1:20 or 1:40 and incubated at room temperature for 15 minutes. To this mixture, 25 μL of DOPE liposomes were added. The transfection mixtures were incubated for 30 minutes at room temperature prior to transfection and added to the cells, dropwise. The plates were incubated for periods of 5 or 6-24 hours at 37° C. in a CO₂ incubator. The transfection mix was replaced with supplemented MEM and the plates further incubated for 24 hours. The supernatants were collected and stored at −20° C. As a positive control, the cells were transfected with Lipofectamine Plus™ Reagent, as described above.

### Example 4

### Topical Formulations

Two topical delivery systems were prepared with the gemini 16-3-16 surfactant. A cationic liposome formulation was prepared with DOPE 10 mg/mL, 1.2 dipalmitoyl-sn-glycero-phosphatidylcholine (DPPC) (Sigma) 10 mg/mL, the gemini 16-3-16 surfactant 10 mg/mL, and diethylene glycol monoethyl ether (Gattefossé, Saint-Priest, France) 25 mg/mL, containing 25 μg of plasmid in 50 μL of formulation (FIG. 2B). A cationic nanoemulsion (all ingredients from Gattefossé) was prepared with PEG-8 caprylic/capric glycerides 200 mg/mL, polyglyceryl-3-isostearate 200 mg/mL, octyldodecyl myristate 400 mg/mL and the gemini 16-3-16 surfactant 10 mg/mL (FIG. 2C). As a control, cholesteryl 3β-(-N-[dimethylamino-ethyl]carbamate) (Dc-chol, Sigma) was used in liposomal formulation: DOPE 10 mg/mL, DPPC 10 mg/mL, Dc-chol 10 mg/mL, and diethylene glycol monoethyl ether (Gattefossé) 25 mg/mL, containing 25 μg of plasmid in 50 μL of formulation. The plasmid concentration was 25 μg in 50 μL formulation. The formulations were characterized (by visual appearance, atomic force microscopy and pH).

### Example 5

### Effects of Topical Formulations of Gemini Cationic Surfactants in a Murine Model

a) Treatment Groups

A murine model was used to examine the effects of topical formulations of the gemini cationic surfactant. The animal experiments were approved by the University Committee on Animal Care and Supply Protocol Review Committee. CD1 mice were obtained from the Animal Resource Center, University of Saskatchewan, Saskatoon, SK. The mice were anesthetized with isoflurane and close-shaved the day prior to treatment. For the topically treated groups, the animals were anesthetized with acepromazine 2 mg/kg and ketamine 100 mg/kg injected intraperitnoneally, the shaved area was cleaned with distilled water using sterile gauze and dried. Liposomal and microemulsion formulations (50 μL containing 25 μg pGTmCMV.IFN-GFP plasmid for each animal) were painted on the shaved area, using a pipette, and covered with Parafilm™ for 2-3 hours. Treatments were repeated at 24 and 48-hour intervals. Each animal received a total dose of 75 μg pGTmCMV.IFN-GFP plasmid. In the injected groups, the animals were intradermally treated on their backs at three locations, with liposomal plasmid formulation (5 μg plasmid/site), low and high concentration of aqueous plasmid solution (2.5 and 5 μg plasmid/site) or phosphate buffered saline (PBS). The total dose for the animals injected with liposomal plasmid formulation was 15 μg pGTmCMV.IFN-GFP/animal and for the injected DNA solutions 7.5 μg plasmid (low dose) and 15 μg plasmid/animal (high dose). The naive animals were anesthetized and left untreated. Blood samples, skin biopsies and lymph nodes from inguinal and axillary sites were taken 24 h after the last treatment.

b) Sample Processing

Plasma was separated by centrifugation from blood collected from animals by cardiac puncture, and stored at −20° C. The skin was rinsed with distilled water using sterile gauze and the treated areas were excised. The samples were snap-frozen in liquid nitrogen, and stored at −80° C. The axillary and inguinal lymph nodes were collected, snap-frozen in liquid nitrogen, and stored at −80° C. The skin was homogenized under liquid nitrogen using a biopulverizer. The device was thoroughly cleaned, disinfected with 70% ethanol and dried in a laminar flow hood, under UV light for 15 minutes between the recovery from different groups to avoid cross-contamination. The lymph nodes were homogenized in microfuge tubes with disposable pellet pestles (Kontes, VWR, Mississauga, ON). The homogenized skin samples dedicated for ELISA and the lymph nodes were resuspended in 500 μL protein resuspension buffer (PBS containing leupeptin 10 μg/mL (Sigma) and soybean trypsin inhibitor 20 μg/mL (Sigma)). The homogenates were vortexed for 1 minute, sonicated for 30 seconds and kept on ice for 1 minute. This cycle was repeated three times. All samples were centrifuged at 16,000 g for 15 minutes. The supernatants, free of cell debris, were collected and stored at −20° C. The homogenized skin samples for PCR were resuspended in proteinase K solution (200 μg/mL), incubated for 2 hours at 56° C., boiled for 10 minutes, chilled on ice for 10 minutes and centrifuged at 16000 g at 4° C. for 20 minutes. The supernatants were collected and used for PCR.

### Example 6

### Enzyme-Linked Immunosorbent Assay (ELISA)

ELISAs were performed using round bottom 96-well plates (Immulon II, Dynatech Laboratories, Chantilly, Va.). The plates were coated with 50 μL/well of capture antibody, rat anti-mouse IFNγ (Pharmingen, Mississauga, ON) 2 μg/mL coating buffer and incubated for 24 hours at 4° C. The wells were blocked with 1% bovine serum albumin (BSA) (New England Biolabs, Mississauga, ON) solution in PBS at room temperature for one hour. IFNγ standard (Pharmingen) of 250-2000 pg/mL concentration and was used in 1% BSA solution on plates. Protein resuspension buffer was used as a blank control. The supernatants from cell cultures and the skin and lymph homogenates, as well as the serum from mice, were diluted one in four on the plates and incubated overnight at 4° C. Biotinylated rat anti mouse IFNγ (Pharmingen) was added at 0.5 ng/mL concentration in 1% BSA solution. The plates were incubated for a further 2 h at room temperature. The streptavidin-alkaline phosphatase conjugate (Jackson Immuno Research Laboratories, Inc., West Grove, Pa.) was added in 1:5000 dilution and incubated for 1 h at room temperature, followed by addition of 4-nitrophenyl phosphate di(tris) salt 1 mg/mL in PNPP buffer (1% diethanolamine, 0.5 mM MgCl₂, pH 9.8) (Sigma). Optical density of the samples was measured at 405 nm using a Benchmark Microplate Reader (BioRad, Mississauga, ON). The concentration of the IFNγ vas calculated from the standard IFNγ curve, using recombinant murine IFNγ (Pharmingen).

### Example 7

### Antiviral Assay for Testing the Activity of IFNγ

Murine IFNγ was tested for biological activity based on the reduction of the viral cytopathic effect. L929 cells (ATTC# CCL-1) were plated on a 96-well flat bottom plate (Greiner Labortechnik GmbH, Germany) (5×10⁴ cells/well) in RPMI-1640 medium (GIBCO BRL) supplemented with 10% FBS, 0.1 mM 2-mercaptoethanol (Sigma), 0.8 mM sodium pyruvate (Sigma) and Antibiotic Antimycotic Solution (Sigma). Supernatants from PAM 212 cells transfected with pGTmCMV.IFN-GFP and containing expressed IFNγ were added to the wells in serial dilution. The plates were incubated for 24 hours at 37° C., 5% CO₂. The media were replaced with a 100-fold greater titer of endomyocarditis (EMC) virus (Familletti, et al.) in 100 μL media, and incubated overnight at 37° C., 5% CO₂. The wells were washed with PBS and the cells fixed for 15 minutes in 4% formaldehyde (Sigma) and stained with 0.05% crystal violet (Sigma) solution in 20% methanol for 15 minutes. The plates were then washed and dried. Before measurements were carried out, 100 μL methanol/well was added to the plates. The absorbance was read at 595 nm on an automated plate reader (PowerWaveₓ, Biotech Instruments Inc., Winooski, Vt.). To verify the non-specific antiviral activity, monoclonal antibody against IFNγ activity XMG1.2 (Cherwinski, et al.) was added to the supernatants. Recombinant murine IFNγ standard (Pharmingen) was also included in the assay.

### Example 8

### Fluorescent and Atomic Force Microscopy

Cells were grown in 24-well plates on cover slips and transfected with plasmids, as described. Twenty four hours after transfection, the cells were washed twice with PBS and cover slips mounted. Skin samples snap-frozen in liquid nitrogen were embedded in Tissue-Tek O.C.T. Compound (Canemco, St. Laurent, QB), and cut in 7-μm thick sections. They were mounted on poly-D-lysine-coated microscope slides. Phase contrast and fluorescent images were registered using Axiovert 200M inverted microscope (Zeiss, Germany), with LD-A Plan 40X objective lens. The excitation wavelength for GFP was 488 nm and emission wavelength 507 nm (FITC filter). Autofluorescence was detected with rhodamine filter (excitation at 570 nm, emission at 590 nm).

Atomic force microscopy images were obtained by Pico SPM instrument (Molecular Imaging Inc., Tempe, Ariz.), with MAC-mode, using MI MAC cantilever Type II (K=1.2-5.5 N/m). The DNA-gemini-DOPE liposomes, topical liposomal formulation or nanoemulsion (10 μl each) were spread on the surface of freshly cleaved mica (Grade V-4, SPI Supplies, West Chester, Pa.), and incubated for 15 minutes at room temperature. The excess of formulation was removed with lint free absorbent tissue, and the surface dried with N₂. A 4×4 μm area was scanned for the DNA-gemini-DOPE liposomes, and nanoemulsion, and a 35×35 μm area for the topical liposome formulation.

### Example 9

### Size Measurement

Aqueous solution of pGTmCMV.IFN-GFP plasmid was prepared at 500 μg/mL concentration. Transfection mixture with the pGTmCMV.IFN-GFP plasmid, 16-3-16 gemini surfactant (1:10 charge ratio) and DOPE vesicles was prepared as described earlier for transfection of PAM212 keratinocytes. Control mixture was prepared by replacing the plasmid DNA with water. Topical liposomal formulation using the 16-3-16 gemini surfactant and nanoemulsion formulation using the 16-3-16 surfactant were prepared as described in Example 4. Blank formulations, without plasmid were also prepared. The size of the particles was measured with Nano ZS instrument (Malvern Instruments, Worchestershire, UK).

### Example 10

### Fluorescence-Activated Cell Sorter (FACS)

PAM212 cells were seeded on 6-well plates (Costar, Corning N.Y.) at a density of 1×10⁶ cells/well density and grown to 60-80% confluency. The supplemented MEM was changed to MEM one hour prior to transfection. The transfection mixes were prepared with 1 μg pGTmCMV.IFN-GFP, using the 16-3-16 gemini cationic surfactant and DOPE liposomes at plasmid DNA:gemini cationic surfactant charge ratio of 1:5, 1:10, 1:20 or 1:40, as described earlier, keeping the concentration of the reagents constant. The cells were detached using Versene solution containing 0.05% trypsin (Sigma), pelleted at 4° C. and 1000 g for 5 minutes, washed twice with PBS and resuspended in Fa-cola (10 mM PBS pH 7.2, 0.2% gelatin, 0.03% sodium azide). Triplicate samples were pooled. The cell sorter was calibrated with non-transfected cells and 10⁴ cells of each sample were counted.

### Example 11

### Circular Dichroism

Aqueous gemini cationic surfactant solutions and DOPE liposomes were prepared as for in vitro transfection and degassed at 37° C. in a bath sonicator. Plasmid pGTmCMV.IFN-GFP (20 μg/mL) and gemini cationic surfactant were mixed at DNA:gemini cationic surfactant charge ratio of 1:10, using water or DOPE liposomes as a vehicle. The samples were incubated for 10 min at room temperature prior to measurement. Spectra were recorded by using an Applied Photo Physics π* 180 instrument (Leatherhead, UK) with a 1-nm slit, at 37° C.

### Example 12

### Polymerase Chain Reaction (PCR)

Four primers were designed for nested PCR, amplifying a fragment from the pGTmCMV backbone. The external primers were: sense (pKanEF) 5-ACT CAC CGA GGC AGT TCC AT-3′ (SEQ ID NO: 1) and antisense (pKanER) 5′-GGT AGC GTT GCC AAT GAT GT-3′(SEQ ID NO: 2), amplifying a 540-bp fragment of the pGTmCMV.IFN-GFP plasmid. The internal primers were: sense (pKanIF) 5′-ATG GCA AGA TCC TGG TAT CG-3′ (SEQ ID NO: 3) and antisense (pKanIR) 5′-TTA TGC CTC TTC CGA CCA TC-3′ (SEQ ID NO: 4), which amplified a 459-bp fragment from the previous reaction. Standard dilutions were prepared with the pGTmCMV.IFN-GFP plasmid at 10², 10³, 10⁴, 10⁵ and 10⁶ copies/PCR reaction. The PCR mixes were prepared according to the manufacturer's protocol. All primers and PCR Reagent System were purchased from Invitrogen Life Technologies, Carlsbad, Calif. Thirty five μL of supernatant obtained from each skin homogenate was used for amplification with the external primers, and 2 μL of the PCR product was used for amplification with the internal primers. Techne Genius unit (Techne Incorporated, Princeton, N.J.) was used, under the following conditions: hot start for 4 minutes at 94° C., denaturation at 94° C. for 45 seconds, annealing at 56° C. for 30 seconds, extension for 30 seconds at 72° C. and final extension at 72° C. for 7 minutes. The reaction was carried out in thirty cycles. The PCR products were run in 1% agarose gel, the bands stained with ethidium bromide and quantified, based on standard dilutions.

### Example 13

### Statistical Analyses

Statistical analyses included ANOVA (Scheffe's test) and non-parametric test (Kruskal-Wallis test) using SPSS 11.5 for Windows (SPSS Inc., 233 S. Wacker Drive, 11th Floor, Chicago, Ill. 60606).

### Results

i. Plasmid Constructs and Testing of Expression in PAM212 Cells

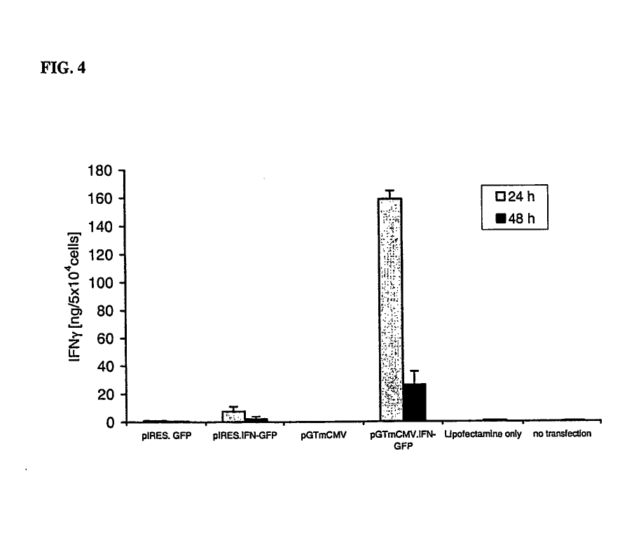

The expression of the IFNγ gene from two plasmid constructs was tested in murine PAM 212 keratinocyte cell culture. FIG. 4 is a graph showing IFNγ expression in PAM212 cells transfected with 0.2 μg pIRES.GFP, pIRES.IFN-GFP, pGTmCMV and pGTmCMV.IFN-GFP, using Lipofectamine Plus™ Reagent. Significant differences were found between the IFNγ expression of Pires.ifh-GFP and pGTmCMV.IFN-GFP (ANOVA p<0.05). The pGTmCMV.IFN-GFP plasmid, having a backbone designed for gene therapy (no CpG motifs), and the murine specific CMV promoter was compared to the pIRES.IFN-GFP plasmid with commercial pIRES2-EGFP backbone. Corresponding control null (non-coding) plasmids were also tested. ELISA test of the cell supernatants showed high expression of IFNγ 24 hours after transfection with the pGTmCMV.IFN-GFP plasmid, whereas significantly lower IFNγ expression was observed with the plasmid based on pIRES backbone (pIRES.IFN-GFP) (p<0.05). Protein expression was at a maximum after 24 hours and, by 48 hours it decreased to about 16% of the 24-hour level.

The IFNγ secreted into the supernatants was biologically active as demonstrated by antiviral assay (Example 7). The average IFNγ concentration was 119.91±39.62 ng/5×10⁵ PAM 212 cells. Preincubation of IFNγ containing supernatants with the XMG1.2 monoclonal antibody against IFNγ resulted in the abolition of cytoprotection. Supernatants from cells incubated with the null plasmids (pIRES and pGTmCMV) did not contain IFNγ nor did they show any antiviral activity (results not shown).

The GFP expression followed the pattern of IFNγ expression with the cells transfected with pGTmCMV.IFN-GFP showing stronger fluorescence than those transfected with pIRES.IFN-GFP (data not shown). The cells treated with null plasmids did not fluoresce.

ii. Optimization of Cationic Gemini Cationic Surfactant—DNA Ratios and Transfection Duration

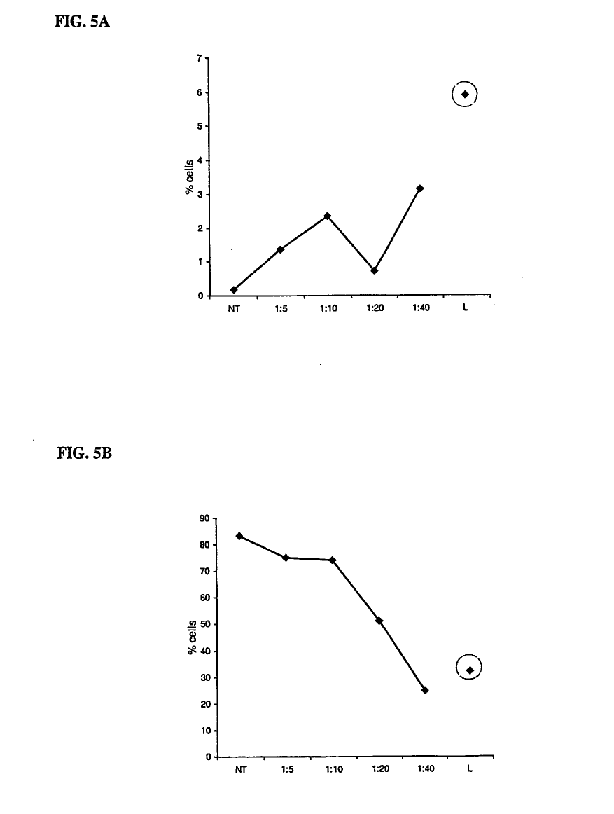

The formulations were optimized for plasmid DNA:gemini surfactant charge ratio correlated with cell toxicity using the 16-3-16 compound. The optimal plasmid DNA:gemini surfactant charge ratio was determined by comparing the percentage of fluorescent cells (FIG. 5A) with cell viability (FIG. 5B). The cells were transfected with either transfection mixtures of the 16-3-16 gemini surfactant and DOPE at plasmid DNA:gemini surfactant charge ratio of 1:5, 1:10, 1:20 or 1:40, or Lipofectamine Plus™ Reagent (L), using 0.2 μg pGTmCMV.IFN-GFP, and compared to non-transfected cells (NT). Triplicate samples were pooled and 1×10⁴ cells counted. A high percentage (2.36%) of fluorescent cells was observed at 1:10 plasmid:gemini surfactant charge ratio, with 74% cell viability. Overall, an increase in the cationic charge ratio with gemini surfactants resulted in increasing transfection efficiency but lower cell viability. At plasmid DNA:gemini surfactant charge ratio of 1:40 the transfection efficiency was 3.13%, however, cell viability fell to 20%. The commercially available Lipofectamine Plus™ Reagent (DOSPA:DOPE 3:1; at the concentration recommended by the manufacturer), used as positive control yielded slightly higher fluorescent cell count (5.87%), but cell viability was only 32%. No fluorescence was detected in the non-transfected cells.

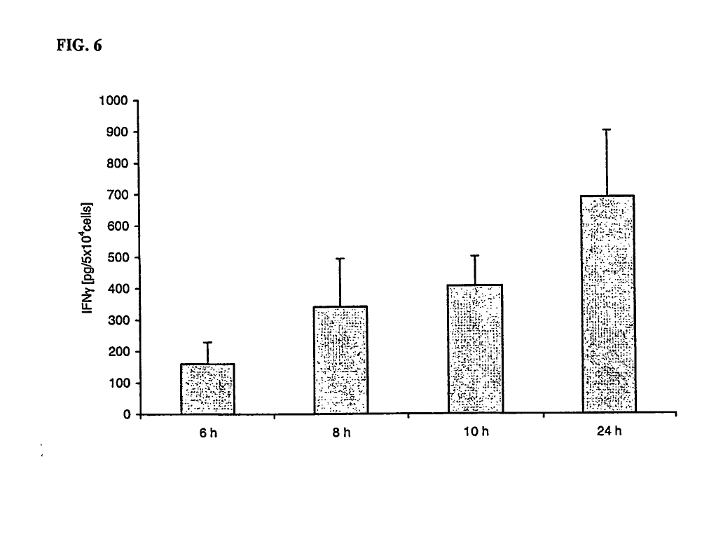

FIG. 6 is a graph showing the influence of transfection duration on the efficiency of the transfection. PAM 212 cells were transfected with 0.2 μg pGTmCMV.IFN-GFP using the transfection mixtures consisting of DOPE and the 16-3-16 gemini surfactant at plasmid DNA:gemini surfactant charge ratio of 1:10 for 6, 8, 10 or 24 hours. The optimum duration of transfection was found to be 24 hours.

iii. Effect of Gemini Cationic Surfactant Series 12-n-12 and a 16-3-16 on the Transfection of PAM212 Cells with pGTmCMV.IFN-GFP Plasmid

In order to select a gemini analogue with the highest in vitro transfection efficiency and for use in the in vivo studies, eight different gemini surfactants (head group spacer length n=3-16, and alkyl chain length of m=12 or 16) were screened in vitro. The following general composition was selected, based on the optimization studies: plasmid-gemini surfactant charge ratio 1:10 in 1 mM DOPE vesicles. The transfection efficiency was found to be dependent on the length of the spacer between the two positively charged head groups, with the C3 spacer showing the highest activity (both 12-3-12 and 16-3-16) (Table 1).

TABLE 1
The magnitude of in vitro transfection correlated with physico-chemical
characteristics of the gemini surfactants.

| | IFN-γ | Physico-chemical parameters | | | | |
|---|---|---|---|---|---|---|
| | expression | | | | | Krafft |
| Gemini | [ng/5 × 10⁵ | CMC^{a} | a^{a} | | Γ^{b} × 10⁶ | point^{c} |
| compound | cells] | [mM] | [nm²/molecule] | α^{a} | [mol/m²] | [° C.] |
| 12-3-12 | 1.11 ± 0.65 | 0.98 ± 0.04 | 0.98 | 0.23 ± 0.02 | 1.58 | 12.7 |
| 12-4-12 | 0.66 ± 0.32 | 1.17 ± 0.04 | 1.10 | 0.26 ± 0.02 | 1.43 | 10.6 |
| 12-6-12 | 0.48 ± 0.17 | 1.09 ± 0.04 | 1.40 | 0.34 ± 0.02 | 1.16 | <0 |
| 12-8-12 | 0.24 ± 0.12 | 0.84 ± 0.03 | 1.78 | 0.46 ± 0.04 | 0.94 | <0 |
| 12-10-12 | 0.29 ± 0.10 | 0.62 ± 0.03 | 2.16 | 0.51 ± 0.06 | 0.75 | <0 |
| 12-12-12 | 0.33 ± 0.16 | 0.36 ± 0.03 | 2.14 | 0.56 ± 0.08 | 0.73 | 13.6 |
| 12-16-12 | 0.49 ± 0.19 | 0.12 ± 0.01 | 1.44 | 0.59 ± 0.08 | 0.83 | 24.3 |
| 16-3-16 | 1.63 ± 1.37 | 0.026 ± 0.001 | 1.02 | 0.35 ± 0.02 | 1.08 | 42 |

CMC (critical micellar concentration);
α (degree of micelle ionization);
a (head group areas based on activity);
Γ (surface excess concentration);
^{a}from Wettig et al., 2001,
^{b}from Alami et al., 1993,
^{c}from Zana, 2002.

In the 12-n-12 series gemini surfactants, a hyperbolic pattern is noticeable with minimum expression of protein occurring at spacer n=8. Significantly higher IFNγ levels were found when the cells were transfected with 12-3-12, 12-4-12 and 16-3-16 versus the other compounds with longer linkers (p<0.001). No IFNγ was detected when the cells were transfected with DOPE or the 12-n-12 gemini surfactant, alone, nor with plasmid without transfection agents.

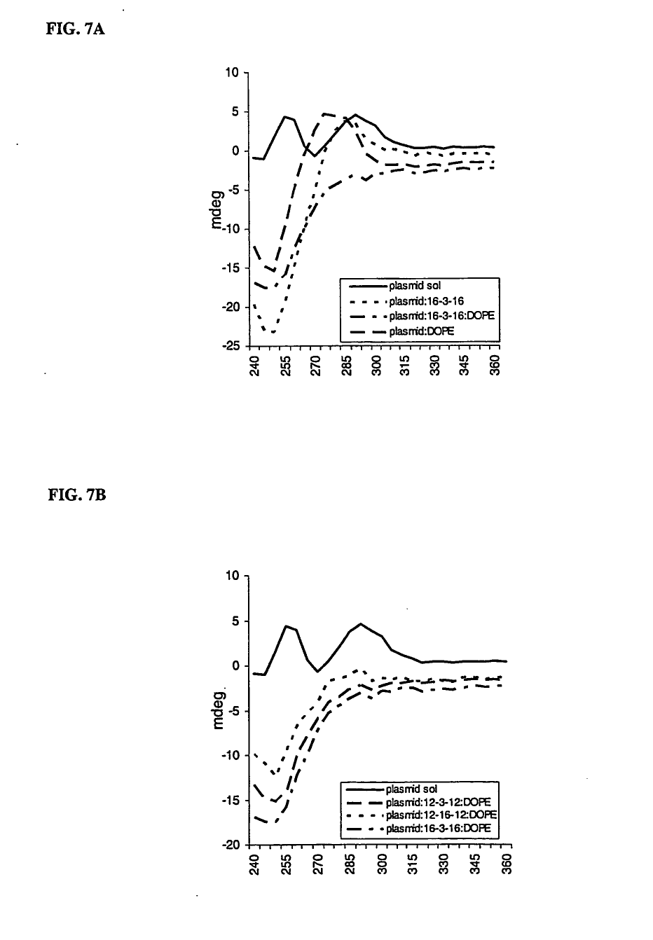

FIG. 7A illustrates the circular dichroism spectra of the pGTmCMV.IFN-GFP plasmid 20 μg/mL in water, in DOPE suspension or coupled with 16-3-13 at plasmid DNA:gemini surfactant charge ratio of 1:10 in water, and in DOPE suspension. FIG. 7B illustrates the circular dichroism spectra of the pGTmCMV.IFN-GFP plasmid 20 μg/mL in water, DNA-12-3-12-DOPE liposomes or DNA-12-16-12-DOPE liposomes, and DNA-16-3-16-DOPE liposomes. Circular dichroism (CD) indicated structural changes in the DNA structure, induced by the gemini cationic surfactant/DOPE liposomes. CD spectra show that only the gemini surfactant/DOPE liposomes decrease the positive peak at 290 nm and shift the 260 nm peak to negative values (FIG. 7A), whereas gemini surfactant alone or DOPE alone do not induce these changes. All three combinations, i.e. the gemini surfactants or DOPE, alone, or gemini surfactant—DOPE liposomes induced a shift of the 260 nm peak to negative values in the spectrum of DNA. The pattern changes induced by the 12-carbon series was similar, showing a peak (in the negative region) at 240-250 nm (FIG. 7B). This peak was higher for the 16-carbon spacer than the 3-carbon spacer.

The transfection mixtures with the 16-3-16 gemini compound, having the highest transfection efficiency were characterized by atomic force microscopy (data not shown) and size measurement (FIG. 3A) as having average particle size of 100-200 nm. TR-16 is the blank transfection mixture with the 16-3-16 surfactant; DNA-TR16 is the transfection mixture used for transfection of PAM212 cells with the pGTmCMV.IFN-GFP plasmid.

iv. Topical Transfection of pGTmCMV.IFN-GFP Plasmid Using Gemini Cationic Surfactant (16-3-16)-Lipid Systems

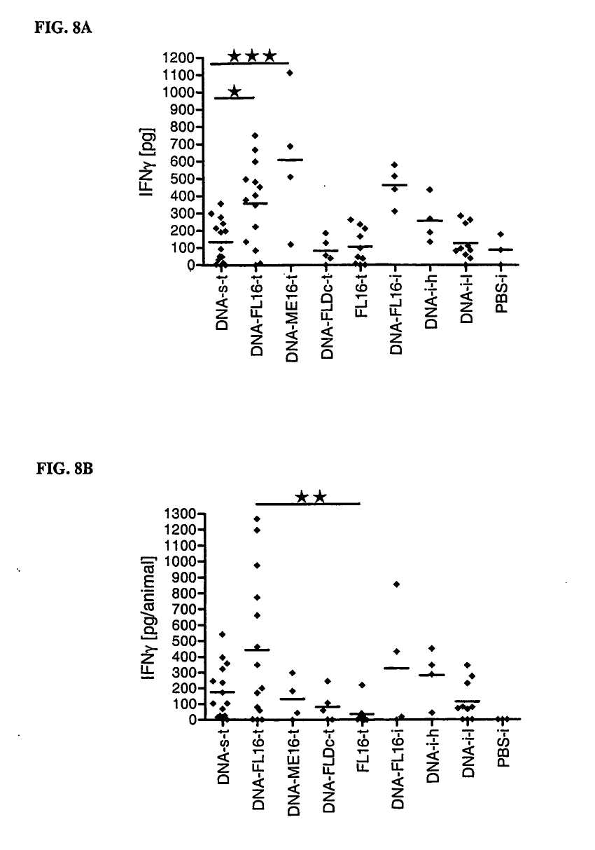

FIG. 8A is a graph showing IFNγ expression in skin in the mice treated with the pGTmCMV.IFN-GFP plasmid and gemini lipid 16-3-16 in various formulations. Results are expressed as amount of IFNγ/cm² treated skin for the topical treatment and as amount of IFNγ/animal for the injected groups. FIG. 8B is a graph showing IFNγ expression in lymph nodes in the mice treated with the pGTmCMV.IFN-GFP plasmid and gemini lipid 16-3-16 in various formulations. Results are expressed as amount of IFNγ/animal for all groups. Results were combined from four experiments and standardized by subtracting the background values obtained in the naive animals in each experiment from the other groups. Significant differences were observed at the p<0.05 level (ANOVA). The groups and treatments are set out below:

| Group | Treatment |
|---|---|
| DNA-s-t (n = 15) | topically treated with pGTmCMV.IFN-GFP 25 μg in 50 μL in |
| | aqueous solution for three days (total dose 75 μg) |
| DNA-FL16-t (n = 14) | topically treated with pGTmCMV.IFN-GFP in cationic gemini |
| | liposomal formulation 25 μg in 50 μL for three days (total dose 75 μg) |
| DNA-ME16-t (n = 4) | topically treated with pGTmCMV.IFN-GFP in cationic gemini |
| | nanoemulsion formulation 25 μg in 50 μL for three days (total dose |
| | 75 μg) |
| DNA-FLDc-t (n = 5) | topically treated with pGTmCMV.IFN-GFP in Dc-chol liposomal |
| | formulation 25 μg in 50 μL for three days (total dose 75 μg) |
| FL16-t (n = 10) | topically treated with 50 μL mock cationic gemini liposomal |
| | formulation for three days |
| DNA-FL16-i (n = 4) | intradermally injected with pGTmCMV.IFN-GFP in cationic |
| | gemini liposomal formulation 5 μg in 10 μL (total dose 15 μg) for |
| | one day |
| DNA-i-h (n = 4) | intradermally injected with pGTmCMV.IFN-GFP in aqueous |
| | solution (5 μg in 10 μL, total dose 15 μg) for one day |
| DNA-i-1 (n = 10) | intradermally injected with pGTmCMV.IFN-GFP in aqueous |
| | solution (2.5 μg in 10 μL, total dose 7.5 μg) for one day |
| PBS-i (n = 3) | intradermally injected with 10 μL PBS for one day |

Generally, topical treatment of mice with pGTmCMV.IFN-GFP plasmid in liposomal or nanoemulsion lipid formulations resulted in high levels of IFNγ expression in the skin (FIG. 8A) and lymph nodes (FIG. 8B). Topical application of gemini cationic liposomal DNA (3×25 μg DNA total dose) and nanoemulsion formulation (3×25 μg DNA total dose) lead to significantly higher IFNγ expression in the skin than topical naked DNA and blank liposomal formulation (359.4 and 607.24 compared to 139.69 and 82.15 pg IFNγ/cm²). The IFNγ levels in the skin of animals treated topically with naked DNA (139.69 pg IFNγ/cm²) or Dc-chol formulation (82.15 pg IFNγ/cm²) were not statistically different from the control group treated with placebo liposomes (105.87 pg IFNγ/cm²).

IFNγ expression in the lymph nodes was the highest in the animals treated topically with gemini liposomal formulations, at significantly higher levels compared to the control gemini liposomes (442.74 vs 35.74 pg/animal) (p<0.05). Application of the 16-3-16 gemini liposomal formulation induced four fold higher levels of IFNγ than the Dc-chol formulation in the lymph nodes. Dc-chol was selected as a control for the in vivo studies since laboratory (Caplen et al., Nomura et al.) and clinical trials (Gill et al.) showed its ability to deliver plasmid DNA in vivo in various tissues. The use of Lipofectamine Plus™ for the animal experiments was not feasible due to the high plasmid concentration in the topical formulations. The IFNγ expression after intradermal injection of liposomal formulation of the plasmid in gemini liposomes (2.5 μg/10 μL) was approximately three times higher than intradermal injection of naked DNA solution (same dose) in both skin and lymph nodes. In the animals injected intradermally with naked DNA at a dose of 5 μg/10 μL and 2.5 μg/10 μL, respectively, protein expression was proportional with the dose, both in the skin (225.68 pg/cm² vs. 126.7 pg/cm²) and the lymph nodes (281.14 pg/animal vs. 114.73 pg/animal). No IFNγ could be detected in the serum obtained from the animals in any of the groups.

Additionally to IFNγ quantitation, GFP fluorescence was also used to visualize gene expression in the skin. GFP expression was detected in the skin treated with liposomal formulation in the epidermis and around the injection site in skin injected with 2.5 μg DNA/site (data not shown). No GFP fluorescence was observed in the skin of animals treated with control (no DNA) liposomal formulation. Autofluorescence was ruled out by comparing the images to those taken in the rhodamine emission band.

Quantitation of plasmid delivery was carried out by nested PCR in the skin of the animals treated intradermally and topically with the gemini cationic liposomal formulation (Table 2). The ethidium bromide-stained bands were quantified based on standard dilutions of the same plasmid. Approximately 21.04×10⁶ copies/cm² skin were detected after 3×25 μg DNA in gemini liposomes topical treatment (a single treatment resulted in 16.25×10⁶ copies/cm² skin; results not shown). When gemini cationic liposomal formulation of 5 μg DNA/site was injected intradermally, about 134.04×10⁶ copies of DNA/cm² skin. Low plasmid level was detected in the skin of the gemini nanoemulsion treated group (2.5×10⁶ copies/cm²). No plasmid was present in the skin of animals injected with 5 μg DNA solution/site, nor in the control group.

The topical formulations, both the liposomal formulation and nanoemulsion prepared with the 16-3-16 surfactant were characterized by atomic force microscopy (data not shown) and size measurement. The liposomal formulation shows heterogeneous distribution of smaller 100-200 nm particles and larger 2-5 m particles (FIG. 3B, DNAs-pGTmCMV.IFN-GFP plasmid aqueous solution (500 μg/mL); TR16-blank transfection mixture with the 16-3-16 surfactant, DNA-TR16-transfection mixture used for transfection of PAM212 cells with the pGTmCMV.IFN-GFP plasmid). The nanoemulsion formulation contains particles of 5-10 nm (FIG. 3C, DNAs-pGTmCMV.IFN-GFP plasmid aqueous solution (500 μg/mL), ME16-blank nanoemulsion formulation with the 16-3-16 surfactant, DNA-ME16-topical nanoemulsion formulation with the pGTmCMV.IFN-GFP plasmid.

TABLE 2
Nested PCR for pGTmCMV.IFN-GFP plasmid detection in the skin.

| | # copies of |
|---|---|
| Treatment | pGTmCMV.IFN-GFP/cm² skin |
| DNA-L-top (3 × 24 h) (n = 4) | 21.04 ± 18.54 × 10⁶ |
| DNA-ME-top (3 × 24 h) (n = 4) | 2.5 ± 0.01 × 10⁶ |
| DNA-L-inj (1 × 24 h) (n = 3) | 134.04 ± 89.66 × 10⁶ |
| DNA-inj-h (1 × 24 h) (n = 4) | 0 |
| PBS-inj (1 × 24 h) (n = 3) | 0 |

### REFERENCES

Arnett, F. C. (2002) Systemic sclerosis (scleroderma). In The Molecular Pathology of Autoimmune Diseases, Theofilopoulos A N, Constantin A. Bona C A (eds). Taylor & Francis: New York, pp. 453-503.
Ausubel, F. M., Bent, R., Kingston, R. E., Moore, D. J., Smith, J. A., Silverman, J. G. and Struhl K. (2000) Current Protocols in Molecular Biology. John Wiley & Sons, New York.
Babiuk, S., Baca-Estrada, M. E., Pontarollo, R. and Foldvari, M. (2002) Topical delivery of plasmid DNA using biphasic lipid vesicles (Biphasix). J Pharm Pharmacol. 54(12):1609-14.
Barron, L. G., Gagne, L. and Szoka, F. C., Jr. (1999) Lipoplex-mediated gene delivery to the lung occurs within 60 minutes of intravenous administration. Hum Gene Ther. 10(10):1683-94.
Birchall, J. C., Marichal, C., Campbell, L., Alwan, A., Hadgraft, J. and Gumbleton, M. (2000) Gene expression in an intact ex-vivo skin tissue model following percutaneous delivery of cationic liposome-plasmid DNA complexes. Int J Pharm. 197(1-2):233-8.
Caplen, N. J., Kinrade, E., Sorgi, F., Gao, X., Gruenert, D., Geddes, D., Coutelle, C., Huang, L., Alton, E. W. and Williamson, R. (1995) In vitro liposome-mediated DNA transfection of epithelial cell lines using the cationic liposome DC-Chol/DOPE. Gene Ther. 2:603-13.
Chattaraj, S. C. and Walker, R. B. (1995) Penetration enhancer classification. In: Smith, E. W. and Maibach H. I. (eds) Percutaneous penetration enhancers. CRC Press, Boca Raton, F L. pp. 5-20.
Chen, X., Wang, J., Shen, N., et al. (2002) Gemini surfactant/DNA complex monolayers at the air-water interface: Effect of surfactant structure on the assembly, stability, and topography of monolayers. Langmuir 18(16):6222-6228.
Cherwinski, H. M., Schumacher, J. H., Brown, K. D. and Mosmann, T. R. (1987) Two types of mouse helper T cell clone. III. Further differences in lymphokine synthesis between Th1 and Th2 clones revealed by RNA hybridization, functionally monospecific bioassays, and monoclonal antibodies. J Exp Med. 166(5):1229-44.
Delepine, P., Guillaume, C., Floch, V., Loisel, S., Yaouanc, J., Clement, J., Des Abbayes, H., Ferec, C. (2000) Cationic phosphonolipids as nonviral vectors: in vitro and in vivo applications. J Pharm Sci 89(5):629-38.
Dorsch-Hasler, K., Keil, G. M., Weber, F., Jasin, M., Schaffner, W. and Koszinowski, U. H. (1985) A long and complex enhancer activates transcription of the gene coding for the highly abundant immediate early mRNA in murine cytomegalovirus. Proc Natl Acad Sci USA 82(24):8325-9.
Familletti, P. C., Rubinstein, S. and Pestka, S. (1981) A convenient and rapid cytopathic effect inhibition assay for interferon. Methods Enzymol 78(9):387-94.
Fan, H., Lin, Q., Morrissey, G. R. and Khavari, P. A. (1999) Immunization via hair follicles by topical application of naked DNA to normal skin. Nat Biotechnol 17(9):870-2.
Friel. J. P. (ed.) (1974) Dorland's Illustrated Medical Dictionary, 25^{th} Edition, W. B. Saunders Company, Philadelphia, pp. 425.
Galperin, C. and Gershwin, M. E. (1998) Systemic sclerosis (scleroderma). In The Autoimmune diseases, Rose, N. R. and Mackay I. R. (eds). Academic Press: San Diego, pp. 317-342.
Gill, D. R., Southern, K. W., Mofford, K. A., Seddon, T., Huang, L., Sorgi, F., Thomson, A., MacVinish, L. J., Ratcliff, R., Bilton, D., Lane, D. J., Littlewood, J. M., Webb, A. K., Middleton, P. G., Colledge, W. H., Cuthbert, A. W., Evans, M. J., Higgins, C. P. and Hyde, S. C. (1997) A placebo-controlled study of liposome-mediated gene transfer to the nasal epithelium of patients with cystic fibrosis. Gene Ther. 4: 199-209.
Gray, P. W. and Goeddel, D. V. (1983) Cloning and expression of murine immune interferon cDNA. Proc Natl Acad Sci USA 80(19):5842-6.
Harrop, A. R., Ghahary, A., Scott, P. G., Forsyth, N., Uji-Friedland, A. and Tredget, E. E. (1995) Regulation of collagen synthesis and mRNA expression in normal and hypertrophic scar fibroblasts in vitro by interferon-gamma. J Surg Res 58(5):471-7.
Katsunuma, T., Kawahara, H., Yuki, K., Akasawa, A. and Saito, H. (2004) Impaired interferon gamma production in a subset population of severe atopic dermatitis. Int. Arch. Allergy Immunol. 134(3):240-7.
Kirby, A. J., Camilleri, P., Engberts, J. B. F. N., Feiters, M. C., Nolte, R. J. M., Soderman, O., Bergsma, M., Bell, P. C., Fielden, M. L., Rodriguez, C. L. G., Guedat, P., Kremer, A., McGregor, C., Perrin, C., Ronsin, G. and van Eijk, M. C. P. (2003) Gemini surfactants: new synthetic vectors for gene transfection. Angew. Chem. Int. Ed. 42:1448-1457.
Lewis, P. J., Cox, G. J., van Drunen Littel-van den Hurk, S. and Babiuk, L. A. (1997) Polynucleotide vaccines in animals: enhancing and modulating responses. Vaccine 15(8):861-4.
Luger, T. A., Beissert, S. and Schwarz, T. (1997) The epidermal cytokine network. In Skin Immune System (SIS), Bos ID (ed). CRC Press: Boca Raton; pp. 271-310.
Menger, F. M. and Keiper, J. S. (2000) Gemini surfactants. Angew. Chem. Int. Ed. 39:1906-1920.
Moschella, S. L. and Hurley, H. J. (1992) Connective tissue diseases. In Dermatology, Moschella S L, Hurley H J (eds). W. B. Saunders Company: Philadelphia, pp. 1233-1245.
Nomura, T., Yasuda, K., Yamada, T., Okamoto, S., Mahato, R. I., Watanabe, Y., Takakura, Y. and Hashida, M. (1999) Gene expression and antitumor effects following direct interferon (IFN)-gamma gene transfer with naked plasmid DNA and DC-chol liposome complexes in mice. Gene Ther. 6: 121-9.
Sambrook, J., Fritsch, E. F. and Maniatis, T. (1989) Molecular Cloning, A Laboratory Manual. Cold Spring Harbour Laboratory Press.
Spirito, F., Meneguzzi, G., Danos, O. and Mezzina, M. (2001) Cutaneous gene transfer and therapy: the present and the future. J Gene Med. 3(1):21-31.
Uitto, J. and Pulkkinen, L. (2000) The genodermatoses: candidate diseases for gene therapy. Human Gene Therapy 11:2267-2275.
Vogel, J. C. (2000) Nonviral skin gene therapy. Human Gene Therapy 11:2253-2259.
Wettig, S. D. and Verrall, R. E. (2001) Thermodynamic Studies of Aqueous m-s-m Gemini Surfactant Systems. J Colloid Interface Sci 235(2):310-316.
Widorn, R. L. (2000) Regulation of matrix biosynthesis and degradation in systemic sclerosis. Curr Opin Rheumatol 12(6):534-9.
Williams, A. C. and Barry, B. W. (1992) Skin absorption enhancers. Crit Rev Ther Drug Carrier Syst. 9(3-4):305-53.
Xu, L., Pirollo, K. F., Tang, W. H., Rait, A. and Chang, E. H. (1999) Transferrin-liposome-mediated systemic p53 gene therapy in combination with radiation results in regression of human head and neck cancer xenografts. Hum Gene Ther 10(18):2941-52.
Zana, R. (2002) Alkanediyl-α,ω-bis(dimethylalkylammonium bromide) Surfactants: II. Krafft Temperature and Melting Temperature. J Colloid Interface Sci 252:259-261.
Zana, R. (2002) Dimeric (Gemini) surfactants: effect of the spacer group on the association behaviour in aqueous solution. J Colloid Interface Sci 248:203-220.

### PATENT DOCUMENTS

Camilleri, P., Kirby, A. J., Perrin, C. and McGregor, C. Novel compounds. United States Patent Application Publication No. US 2003/0119188, published Jun. 26, 2003.
Camilleri, P., Kremer, A. and Rice, S. Q. J. Peptide-based gemini compounds. U.S. Pat. No. 6,693,167, issued Feb. 17, 2004.
Camilleri, P., Kremer, A. and Rice, S. Q. J. Peptide-based gemini compounds. United States Patent Application Publication No. US 2004/0043939, published Mar. 4, 2004.
Camilleri, P., Kirby, A. J., Perrin, C., Ronsin, G. A. B. and Guedat, P. Novel compounds. United States Patent Application Publication No. US 2004/0138139, published Jul. 15, 2004.

All publications mentioned in this specification are indicative of the level of skill in the art to which this invention pertains. All publications are herein incorporated by reference to the same extent as if each individual publication was specifically and individually indicated to be incorporated by reference. Although the foregoing invention has been described in some detail by way of illustration and example, for purposes of clarity and understanding it will be understood that certain changes and modifications may be made without departing from the scope or spirit of the invention as defined by the following claims.

We claim:

1. A delivery system for a biologically active agent comprising:
a gemini surfactant in admixture with a biologically active agent in a topical formulation, wherein the delivery system, when in contact with the skin or mucosal membrane, releases the biologically active agent in a therapeutically-effective amount to provide a localized or systemic effect for treatment of a skin disorder or a metabolic disease.

2. The delivery system according to claim 1, wherein the gemini surfactant is selected from an anionic gemini surfactant, a gemini cationic surfactant, a neutral gemini surfactant, an amphoteric gemini surfactant, or mixtures thereof.

3. The delivery system according to claim 2, wherein the gemini surfactant is a gemini cationic surfactant.

4. The delivery system according to claim 3, wherein the gemini cationic surfactant is of a quaternary ammonium type.

5. The delivery system according to claim 4, wherein the gemini cationic surfactant has a hydrophobic tail comprising a C₃-C₃₀ alkyl group, linear or branched, saturated or unsaturated.

6. The delivery system according to claim 1 or 5, wherein the biologically active agent is selected from the group consisting of a nucleic acid, plasmid DNA, DNA vaccine, protein, vaccine, immunoglobulin, immunomodulator, oligonucleotide, peptide, hormone, toxin, and enzyme.

7. The delivery system according to claim 6, wherein the biologically active agent is selected from the group consisting of a nucleic acid, plasmid DNA, DNA vaccine, and oligonucleotide.

8. The delivery system according to claim 7, wherein the biologically active agent is a plasmid DNA comprising the gene encoding for interferon-γ for treatment of scleroderma, atopic dermatitis, or any condition characterized by interferon-γ deficiency.

9. The delivery system according to claim 1 or 7, wherein the skin disorder is selected from the group consisting of scleroderma, atopic dermatitis, psoriasis, conditions characterized by any cytokine deficiency, conditions characterized by IFNγ deficiency, an epidermal fragility disorder, a keratinization disorder, a hair disorder, a pigmentation disorder, a porphyria, a multisystem disorder, cancer, inherited epidermolysis bullosa; lamellar ichthyosis, X-linked ichthyosis, and xeroderma pigmentosum.

10. The delivery system according to claim 9, wherein the skin disorder is selected from scleroderma, atopic dermatitis, or any condition characterized by interferon-γ deficiency.

11. The delivery system according to claim 1 or 7, wherein the metabolic disease is selected from the group consisting of conditions characterized by any cytokine deficiency, conditions characterized by IFNγ deficiency, gyrate atrophy, maternal hyperphenylalaninemia, familial hypercholesterolemia, and phenylketonuria.

12. The delivery system according to claim 1 or 7, wherein the delivery system is provided in as a coating on, or filler in, a dressing, packing, film, mesh or transdermal patch, or in a pharmaceutical composition with one or more pharmaceutically acceptable carriers, diluents, excipients, or supplements suitable for application to the skin or mucosal membrane.

13. The delivery system according to claim 12, wherein the pharmaceutical composition is in the form of a cream, lotion, paste, ointment, foam, gel, lipid formulation, emulsion, or suspension, or a liquid formulated as an aqueous solution, non-aqueous solution, aerosol, mist, spray, drops, or instillation.

14. The delivery system according to claim 13, wherein the one or more supplements are selected from a neutral carrier or a permeation enhancer.

15. The delivery system according to claim 14, wherein the neutral carrier is selected from dioleyl phosphatidylethanolamine or cholesterol.

16. The delivery system according to claim 14, wherein the permeation enhancer is selected from ethanol, salicylic acid, diethylene glycol monoethyl ether, propylene glycol, oleic acid, a terpene, a surfactant, a fatty acid, a fatty acid ester, azone, an azone derivative, an amide, dimethylformamide, or a sulfoxide.

17. The delivery system according to claim 12, wherein the pharmaceutical composition further comprises one or more surfactants, co-surfactants, or oily phase components suitable for application to the skin or mucosal membrane.

18. The delivery system according to claim 17, wherein the surfactant is selected from the group consisting of PEG-8 caprylic and capric glycerids.

19. The delivery system according to claim 17, wherein the co-surfactant is selected from the group consisting of polyglyceryl 3-diisostearate, polyglyceryl-6 isostearate, polyglycerol-3-isosterate and polyglyceryl-6 dioleate.

20. The delivery system according to claim 17, wherein the oily phase component is selected from the group consisting of propylene glycol monocaprylate, oleoyl macrogol-6 glycerides, PEG-8 glyceryl linoleate, propylene glycol laurate, propylene glycol monolaurate, and octyldodecyl myristate.

21. A pharmaceutical composition in a topical formulation comprising:
the delivery system according to claim 1, in admixture with one or more pharmaceutically acceptable carriers, diluents, excipients, or supplements suitable for application to the skin or mucosal membrane.

22. The pharmaceutical composition according to claim 21, wherein the gemini surfactant is selected from an anionic gemini surfactant, a gemini cationic surfactant, a neutral gemini surfactant, an amphoteric gemini surfactant, or mixtures thereof.

23. The pharmaceutical composition according to claim 22, wherein the gemini surfactant is a gemini cationic surfactant.

24. The pharmaceutical composition according to claim 23, wherein the gemini cationic surfactant is of a quaternary ammonium type.

25. The pharmaceutical composition according to claim 24, wherein the gemini cationic surfactant has a hydrophobic tail comprising a C₃-C₃₀ alkyl group, linear or branched, saturated or unsaturated.

26. The pharmaceutical composition according to claim 21 or 25, wherein the biologically active agent is selected from the group consisting of a nucleic acid, plasmid DNA, DNA vaccine, protein, vaccine, immunoglobulin, immunomodulator, oligonucleotide, peptide, hormone, toxin, and enzyme.

27. The pharmaceutical composition according to claim 26, wherein the biologically active agent is selected from the group consisting of a nucleic acid, plasmid DNA, DNA vaccine, and oligonucleotide.

28. The pharmaceutical composition according to claim 27, wherein the biologically active agent is a plasmid DNA comprising the gene encoding for interferon-γ for treatment of scleroderma, atopic dermatitis, or any condition characterized by interferon-γ deficiency.

29. The pharmaceutical composition according to claim 21 or 27, wherein the skin disorder is selected from the group consisting of scleroderma, atopic dermatitis, psoriasis, conditions characterized by any cytokine deficiency, conditions characterized by IFNγ deficiency, an epidermal fragility disorder, a keratinization disorder, a hair disorder, a pigmentation disorder, a porphyria, a multisystem disorder, cancer, inherited epidermolysis bullosa; lamellar ichthyosis, X-linked ichthyosis, and xeroderma pigmentosum.

30. The pharmaceutical composition according to claim 29, wherein the skin disorder is selected from scleroderma, atopic dermatitis, or any condition characterized by interferon-γ deficiency.

31. The pharmaceutical composition according to claim 21 or 27, wherein the metabolic disease is selected from the group consisting of conditions characterized by any cytokine deficiency, conditions characterized by IFNγ deficiency, gyrate atrophy, maternal hyperphenylalaninemia, familial hypercholesterolemia, and phenylketonuria.

32. The pharmaceutical composition according to claim 21 or 27, wherein the pharmaceutical composition is in the form of a cream, lotion, paste, ointment, foam, gel, lipid formulation, emulsion, or suspension, or a liquid formulated as an aqueous solution, non-aqueous solution, aerosol, mist, spray, drops, or instillation, or as a coating on, or filler in, a dressing, packing, film, mesh or transdermal patch.

33. The pharmaceutical composition according to claim 21, wherein the one or more supplements are selected from a neutral carrier or a permeation enhancer.

34. The pharmaceutical composition according to claim 33, wherein the neutral carrier is selected from dioleyl phosphatidylethanolamine or cholesterol.

35. The pharmaceutical composition according to claim 33, wherein the permeation enhancer is selected from ethanol, salicylic acid, diethylene glycol monoethyl ether, propylene glycol, oleic acid, a terpene, a surfactant, a fatty acid, a fatty acid ester, azone, an azone derivative, an amide, dimethylformamide, or a sulfoxide.

36. The pharmaceutical composition according to claim 21, wherein the pharmaceutical composition further comprises one or more surfactants, co-surfactants, or oily phase components suitable for application to the skin or mucosal membrane.

37. The pharmaceutical composition according to claim 36, wherein the surfactant is selected from the group consisting of PEG-8 caprylic and capric glycerids.

38. The pharmaceutical composition according to claim 36, wherein the co-surfactant is selected from the group consisting of polyglyceryl 3-diisostearate, polyglyceryl-6 isostearate, polyglycerol-3-isosterate and polyglyceryl-6 dioleate.

39. The pharmaceutical composition according to claim 36, wherein the oily phase component is selected from the group consisting of propylene glycol monocaprylate, oleoyl macrogol-6 glycerides, PEG-8 glyceryl linoleate, propylene glycol laurate, propylene glycol monolaurate, and octyldodecyl myristate.

40. A method of treating skin disorders and metabolic diseases comprising:
contacting the skin or mucosal membrane of a subject with a delivery system comprising a gemini surfactant in admixture with a biologically active agent in a topical formulation, wherein the delivery system, when in contact with the skin or mucosal membrane, releases the biologically active agent in a therapeutically-effective amount to provide a localized or systemic effect for treatment of a skin disorder or a metabolic disease.

41. The method according to claim 40, wherein the gemini surfactant is selected from an anionic gemini surfactant, a gemini cationic surfactant, a neutral gemini surfactant, an amphoteric gemini surfactant, or mixtures thereof.

42. The method according to claim 41, wherein the gemini surfactant is a gemini cationic surfactant.

43. The method according to claim 42, wherein the gemini cationic surfactant is of a quaternary ammonium type.

44. The method according to claim 43, wherein the gemini cationic surfactant has a hydrophobic tail comprising a C₃-C₃₀ alkyl group, linear or branched, saturated or unsaturated.

45. The method according to claim 40 or 44, wherein the biologically active agent is selected from the group consisting of a nucleic acid, plasmid DNA, DNA vaccine, protein, vaccine, immunoglobulin, immunomodulator, oligonucleotide, peptide, hormone, toxin, and enzyme.

46. The method according to claim 45, wherein the biologically active agent is selected from the group consisting of a nucleic acid, plasmid DNA, DNA vaccine, and oligonucleotide.

47. The method according to claim 46, wherein the biologically active agent is a plasmid DNA comprising the gene encoding for interferon-γ for treatment of scleroderma, atopic dermatitis, or any condition characterized by interferon-γ deficiency.

48. The method according to claim 40 or 46, wherein the skin disorder is selected from the group consisting of scleroderma, atopic dermatitis, psoriasis, conditions characterized by any cytokine deficiency, conditions characterized by IFNγ deficiency, an epidermal fragility disorder, a keratinization disorder, a hair disorder, a pigmentation disorder, a porphyria, a multisystem disorder, cancer, inherited epidermolysis bullosa; lamellar ichthyosis, X-linked ichthyosis, and xeroderma pigmentosum.

49. The method according to claim 48, wherein the skin disorder is selected from scleroderma, atopic dermatitis, or any condition characterized by interferon-γ deficiency.

50. The method according to claim 40 or 46, wherein the metabolic disease is selected from the group consisting of conditions characterized by any cytokine deficiency, conditions characterized by IFNγ deficiency, gyrate atrophy, maternal hyperphenylalaninemia, familial hypercholesterolemia, and phenylketonuria.

51. The method according to claim 40 or 46, wherein the delivery system is provided in as a coating on, or filler in, a dressing, packing, film, mesh or transdermal patch, or in a pharmaceutical composition with one or more pharmaceutically acceptable carriers, diluents, excipients, or supplements suitable for application to the skin or mucosal membrane.

52. The method according to claim 51, wherein the pharmaceutical composition is in the form of a cream, lotion, paste, ointment, foam, gel, lipid formulation, emulsion, or suspension, or a liquid formulated as an aqueous solution, non-aqueous solution, aerosol, mist, spray, drops, or instillation.

53. The method according to claim 52, wherein the one or more supplements are selected from a neutral carrier or a permeation enhancer.

54. The method according to claim 53, wherein the neutral carrier is selected from dioleyl phosphatidylethanolamine or cholesterol.

55. The method according to claim 53, wherein the permeation enhancer is selected from ethanol, salicylic acid, diethylene glycol monoethyl ether, propylene glycol, oleic acid, a terpene, a surfactant, a fatty acid, a fatty acid ester, azone, an azone derivative, an amide, dimethylformamide, or a sulfoxide.

56. The method according to claim 51, wherein the pharmaceutical composition further comprises one or more surfactants, co-surfactants, or oily phase components suitable for application to the skin or mucosal membrane.

57. The method according to claim 56, wherein the surfactant is selected from the group consisting of PEG-8 caprylic and capric glycerids.

58. The method according to claim 56, wherein the co-surfactant is selected from the group consisting of polyglyceryl 3-diisostearate, polyglyceryl-6 isostearate, polyglycerol-3-isosterate and polyglyceryl-6 dioleate.

59. The method according to claim 56, wherein the oily phase component is selected from the group consisting of propylene glycol monocaprylate, oleoyl macrogol-6 glycerides, PEG-8 glyceryl linoleate, propylene glycol laurate, propylene glycol monolaurate, and octyldodecyl myristate.

60. Use of a delivery system in the treatment of a skin disorder or metabolic disease, wherein the delivery system comprises a gemini surfactant in admixture with a biologically active agent in a topical formulation, and the delivery system, when in contact with the skin or mucosal membrane, releases the biologically active agent in a therapeutically effective amount to provide a localized or systemic effect.

61. The use according to claim 60, wherein the gemini surfactant is selected from an anionic gemini surfactant, a gemini cationic surfactant, a neutral gemini surfactant, an amphoteric gemini surfactant, or mixtures thereof.

62. The use according to claim 61, wherein the gemini surfactant is a gemini cationic surfactant.

63. The use according to claim 62, wherein the gemini cationic surfactant is of a quaternary ammonium type.

64. The use according to claim 63, wherein the gemini cationic surfactant has a hydrophobic tail comprising a C₃-C₃₀ alkyl group, linear or branched, saturated or unsaturated.

65. The use according to claim 60 or 64, wherein the biologically active agent is selected from the group consisting of a nucleic acid, plasmid DNA, DNA vaccine, protein, vaccine, immunoglobulin, immunomodulator, oligonucleotide, peptide, hormone, toxin, and enzyme.

66. The use according to claim 65, wherein the biologically active agent is selected from the group consisting of a nucleic acid, plasmid DNA, DNA vaccine, and oligonucleotide.

67. The use according to claim 66, wherein the biologically active agent is a plasmid DNA comprising the gene encoding for interferon-γ for treatment of scleroderma, atopic dermatitis, or any condition characterized by interferon-γ deficiency.

68. The use according to claim 60 or 66, wherein the skin disorder is selected from the group consisting of scleroderma, atopic dermatitis, psoriasis, conditions characterized by any cytokine deficiency, conditions characterized by IFNγ deficiency, an epidermal fragility disorder, a keratinization disorder, a hair disorder, a pigmentation disorder, a porphyria, a multisystem disorder, cancer, inherited epidermolysis bullosa; lamellar ichthyosis, X-linked ichthyosis, and xeroderma pigmentosum.

69. The use according to claim 68, wherein the skin disorder is selected from scleroderma, atopic dermatitis, or any condition characterized by interferon-γ deficiency.

70. The use according to claim 60 or 66, wherein the metabolic disease is selected from the group consisting of conditions characterized by any cytokine deficiency, conditions characterized by IFNγ deficiency, gyrate atrophy, maternal hyperphenylalaninemia, familial hypercholesterolemia, and phenylketonuria.

71. The use according to claim 60 or 66, wherein the delivery system is provided in as a coating on, or filler in, a dressing, packing, film, mesh or transdermal patch, or in a pharmaceutical composition with one or more pharmaceutically acceptable carriers, diluents, excipients, or supplements suitable for application to the skin or mucosal membrane.

72. The use according to claim 71, wherein the pharmaceutical composition is in the form of a cream, lotion, paste, ointment, foam, gel, lipid formulation, emulsion, or suspension, or a liquid formulated as an aqueous solution, non-aqueous solution, aerosol, mist, spray, drops, or instillation.

73. The use according to claim 72, wherein the one or more supplements are selected from a neutral carrier or a permeation enhancer.

74. The use according to claim 73, wherein the neutral carrier is selected from dioleyl phosphatidylethanolamine or cholesterol.

75. The use according to claim 73, wherein the permeation enhancer is selected from ethanol, salicylic acid, diethylene glycol monoethyl ether, propylene glycol, oleic acid, a terpene, a surfactant, a fatty acid, a fatty acid ester, azone, an azone derivative, an amide, dimethylformamide, or a sulfoxide.

76. The use according to claim 71, wherein the pharmaceutical composition further comprises one or more surfactants, co-surfactants, or oily phase components suitable for application to the skin or mucosal membrane.

77. The use according to claim 76, wherein the surfactant is selected from the group consisting of PEG-8 caprylic and capric glycerids.

78. The use according to claim 76, wherein the co-surfactant is selected from the group consisting of polyglyceryl 3-diisostearate, polyglyceryl-6 isostearate, polyglycerol-3-isosterate and polyglyceryl-6 dioleate.

79. The use according to claim 76, wherein the oily phase component is selected from the group consisting of propylene glycol monocaprylate, oleoyl macrogol-6 glycerides, PEG-8 glyceryl linoleate, propylene glycol laurate, propylene glycol monolaurate, and octyldodecyl myristate.

80. Use of a gemini surfactant in the manufacture of a delivery system with a biologically active agent in a topical formulation for treatment of a skin disorder or metabolic disease.

81. The use according to claim 80, wherein the gemini surfactant is selected from an anionic gemini surfactant, a gemini cationic surfactant, a neutral gemini surfactant, an amphoteric gemini surfactant, or mixtures thereof.

82. The use according to claim 81, wherein the gemini surfactant is a gemini cationic surfactant.

83. The use according to claim 82, wherein the gemini cationic surfactant is of a quaternary ammonium type.

84. The use according to claim 83, wherein the gemini cationic surfactant has a hydrophobic tail comprising a C₃-C₃₀ alkyl group, linear or branched, saturated or unsaturated.

85. The use according to claim 80 or 84, wherein the gemini surfactant is admixed in the topical formulation with the biologically active agent selected from the group consisting of a nucleic acid, plasmid DNA, DNA vaccine, protein, vaccine, immunoglobulin, immunomodulator, oligonucleotide, peptide, hormone, toxin, and enzyme.

86. The use according to claim 85, wherein the biologically active agent is selected from the group consisting of a nucleic acid, plasmid DNA, DNA vaccine, and oligonucleotide.

87. The use according to claim 86, wherein the biologically active agent is a plasmid DNA comprising the gene encoding for interferon-γ for treatment of scleroderma, atopic dermatitis, or any condition characterized by interferon-γ deficiency.

88. The use according to claim 80 or 86, wherein the skin disorder is selected from the group consisting of scleroderma, atopic dermatitis, psoriasis, conditions characterized by any cytokine deficiency, conditions characterized by IFNγ deficiency, an epidermal fragility disorder, a keratinization disorder, a hair disorder, a pigmentation disorder, a porphyria, a multisystem disorder, cancer, inherited epidermolysis bullosa; lamellar ichthyosis, X-linked ichthyosis, and xeroderma pigmentosum.

89. The use according to claim 88, wherein the skin disorder is selected from scleroderma, atopic dermatitis, or any condition characterized by interferon-γ deficiency.

90. The use according to claim 80 or 86, wherein the metabolic disease is selected from the group consisting of conditions characterized by any cytokine deficiency, conditions characterized by IFNγ deficiency, gyrate atrophy, maternal hyperphenylalaninemia, familial hypercholesterolemia, and phenylketonuria.

91. The use according to claim 80 or 86, wherein the topical formulation is in the form of a cream, lotion, paste, ointment, foam, gel, lipid formulation, emulsion, or suspension, or a liquid formulated as an aqueous solution, non-aqueous solution, aerosol, mist, spray, drops, or instillation, or as a coating on, or filler in, a dressing, packing, film, mesh or transdermal patch.

92. The use according to claim 91, wherein the topical formulation comprises one or more pharmaceutically acceptable carriers, diluents, excipients, or supplements suitable for application to the skin or mucosal membrane.

93. The use according to claim 92, wherein the one or more supplements are selected from a neutral carrier or a permeation enhancer.

94. The use according to claim 93, wherein the neutral carrier is selected from dioleyl phosphatidylethanolamine or cholesterol.

95. The use according to claim 93, wherein the permeation enhancer is selected from ethanol, salicylic acid, diethylene glycol monoethyl ether, propylene glycol, oleic acid, a terpene, a surfactant, a fatty acid, a fatty acid ester, azone, an azone derivative, an amide, dimethylformamide, or a sulfoxide.

96. The use according to claim 92, wherein the topical formulation further comprises one or more surfactants, co-surfactants, or oily phase components suitable for application to the skin or mucosal membrane.

97. The use according to claim 96, wherein the surfactant is selected from the group consisting of PEG-8 caprylic and capric glycerids.

98. The use according to claim 96, wherein the co-surfactant is selected from the group consisting of polyglyceryl 3-diisostearate, polyglyceryl-6 isostearate, polyglycerol-3-isosterate and polyglyceryl-6 dioleate.

99. The use according to claim 96, wherein the oily phase component is selected from the group consisting of propylene glycol monocaprylate, oleoyl macrogol-6 glycerides, PEG-8 glyceryl linoleate, propylene glycol laurate, propylene glycol monolaurate, and octyldodecyl myristate.
